(19) United States
(12) Patent Application Publication
(10) Pub. No.: US 2024/0277801 A1
(43) Pub. Date: Aug. 22, 2024

(54) AEROSOL COMPOSITION FOR PULMONARY DELIVERY OF FLAGELLIN

(71) Applicants: INSERM (INSTITUT NATIONAL DE LA SANTÉ ET DE LA RECHERCHE MÉDICALE), Paris (FR); CENTRE NATIONAL DE LA RECHERCHE SCIENTIFIQUE (CNRS), Paris (FR); UNIVERSITÉ DE TOURS FRANÇOIS RABELAIS, Tours (FR); UNIVERSITÉ DE LILLE, Lille (FR); INSTITUT PASTEUR DE LILLE, Lille (FR); CENTRE HOSPITALIER REGIONAL UNIVERSITAIRE DE LILLE, Lille (FR)

(72) Inventors: Nathalie HEUZE VOURC'H, Tours Cedex 1 (FR); Alexie MAYOR, Tours Cedex 1 (FR); Jean-Claude SIRARD, Lille Cedex (FR)

(21) Appl. No.: 18/569,392

(22) PCT Filed: Jun. 30, 2022
(86) PCT No.: PCT/EP2022/068149

§ 371 Date: Dec. 12, 2023

(30) Foreign Application Priority Data
Jun. 30, 2021 (EP) 21182877.7

(51) Int. Cl. A61K 38/16; A61K 9/00; A61K 47/02; A61K 47/12; A61K 47/26

(52) U.S. Cl. CPC A61K 38/164; A61K 9/0078; A61K 47/02; A61K 47/12; A61K 47/26

(57) ABSTRACT

The present invention arises from a formulation study in order to define the best excipients including buffer, surfactant, sugar and amino acid to stabilize Flagellin during mesh-nebulization. One of the key factors in stabilising proteins is determining the optimal pH and buffer system to provide adequate solubility and stability and avoid aggregate formation during the aerosolization process. Different formulation have been assayed in order to obtain a stable and soluble aerosol composition comprising flagellin polypeptide in particular considering the choice of the buffering agent, the surfactant and the pH of the liquid formulation comprising the flagellin polypeptide in order to maintain the activity of the flagellin after the aerosolization process. Accordingly, the present invention relates to an aerosol composition comprising droplets comprising a liquid formulation, wherein the liquid formulation comprises a flagellin polypeptide, a buffer (acetate and/or phosphate) and a surfactant (polysorbate). The aerosol composition of the present invention is suitable for the treatment of lung bacterial infections.

### FIELD OF THE INVENTION

The present invention relates to an aerosol composition comprising droplets comprising a liquid formulation, wherein the liquid formulation, comprises a flagellin polypeptide, a buffer (acetate or phosphate) and a surfactant (polysorbate). The aerosol composition of the present invention is suitable for the treatment of lung bacterial infections.

### BACKGROUND OF THE INVENTION

Pulmonary delivery of therapeutic proteins, could represent an attractive, non-invasive alternative to parental delivery. The pulmonary route of administration has proven to be effective in local and systemic delivery of miscellaneous drugs and biopharmaceuticals to treat pulmonary diseases especially lung bacterial infection.

However, administration of proteins, such as polypeptides, to the lungs is associated with many challenges, such as the need for an appropriate formulation of the polypeptides to overcome strong intermolecular/inter-particle interactions and physico-chemical degradation leading, for example, to aggregation and potentially to loss of biological/therapeutic activity and/or safety issues. For example, proteins can be sensitive towards aerosolization-associated shear stress and/or increase in temperature and/or may exhibit decreased stability at the air-liquid interface in an aerosol.

Flagellin is a biological drug of 28kD used as an immunomodulatory in the context of pneumonia. Hence, pulmonary administration by nebulization of Flagellin will allow targeting directly the site on bacterial infection. However, therapeutics proteins such as Flagellin are especially often sensitive to stress produced by the process of aerosolization. Indeed, mesh-nebulization of flagellin, formulated in saline phosphate buffer (PBS) led to high aggregation of the protein.

Accordingly, it was an object of the present invention to identify formulation systems that are suitable for pulmonary delivery of flagellin and that help to maintain stability and activity of the flagellin polypeptide upon aerosolization/nebulization.

### SUMMARY OF THE INVENTION

The present invention relates to aerosol compositions comprising droplets comprising a liquid formulation, wherein the liquid formulation, comprises a flagellin polypeptide, a buffer (acetate or phosphate) and a surfactant (polysorbate). In particular, the present invention is defined by the claims.

In a first aspect, the present invention relates to an aerosol composition comprising droplets comprising a liquid formulation, wherein the liquid formulation comprises
(i) flagellin polypeptide,
(ii) a buffering agent selected from the group consisting of acetate, phosphate and combinations thereof, and
(iii) a surfactant consisting of polysorbate; and
(iv) an aqueous medium; and
wherein the liquid formulation has a pH which is equal to or lower than about 8.

In a second aspect, the present invention relates to a method of preparing an aerosol composition comprising droplets comprising a liquid formulation, said method comprising the steps:
(i) providing a liquid formulation as defined above,
(ii) nebulizing the liquid formulation provided in step (i) by means of a nebulizer, thereby preparing the aerosol.

In a third aspect the present invention relates to the aerosol composition of the invention or the liquid formulation as defined above for use in a method of delivering a flagellin polypeptide to the lungs of a subject, wherein the aerosol composition is administered to the subject by inhalation or the liquid formulation is administered to the subject by inhalation via a nebulizer.

In another aspect, the present invention, to the aerosol composition of the invention or the liquid formulation as defined above, optionally in combination with at least one antibiotic for use in a method of treating or preventing a lung bacterial infection in a subject, wherein the aerosol composition is administered to the subject by inhalation or the liquid formulation is administered to the subject by inhalation via a nebulizer

### DETAILED DESCRIPTION OF THE INVENTION

The present invention arises from a formulation study in order to define the best excipients including buffer, surfactant, sugar and amino acid to stabilize Flagellin during mesh-nebulization. One of the key factors in stabilising proteins is determining the optimal pH and buffer system to provide adequate solubility and stability and avoid aggregate formation during the aerosolization process.

Here, the various components of the formulation were not chosen at random but were selected on the basis of the Generally recognized as safe (GRAS) list which is a United States Food and Drug Administration (FDA) designation that a chemical or substance added to food is considered safe by experts under the conditions of its intended use (https://en.wikipedia.org/wiki/Generally_recognized_as_safe).

Indeed, the various components either were already used in commercial inhalable products (lung) or either used in formulations of therapeutic proteins tested in clinical phase 2 (thus having already shown an acceptable tolerance).

Thus, different formulations have been assayed in order to obtain a stable and soluble aerosol composition comprising flagellin polypeptide in particular considering the choice of the buffering agent, the surfactant and the pH of the liquid formulation comprising the flagellin polypeptide in order to maintain the activity of the flagellin after the aerosolization process. Hence, the flagellin potency on TLR5 activation was maintained after nebulization in aerosol formulation according the present invention (see Example section)

In a first aspect, the present invention relates to an aerosol composition comprising droplets comprising a liquid formulation, wherein the liquid formulation comprises
(i) flagellin polypeptide,
(ii) a buffering agent selected from the group consisting of acetate, phosphate and combinations thereof, and
(iii) a surfactant consisting of polysorbate; and
(iv) an aqueous medium; and
wherein the liquid formulation has a pH which is equal to or lower than about 8.

As used herein, the term “flagellin” has its general meaning in the art and refers to the flagellin contained in a variety of Gram-positive or Gram-negative bacterial species. Non-limiting sources of flagellins include but are not limited to Escherichia, e.g., E. coli, Enterobacter, Erwinia, Klebsiella, Proteus, Salmonella, e.g., Salmonella enterica serovar Typhimurium, Serratia, e.g., Serratia marcescans, and Shigella, as well as Bacilli such as B. subtilis and B. licheniformis, Pseudomonas such as P. aeruginosa, and Streptomyces. These examples are illustrative rather than limiting. The amino acid sequences and nucleotide sequences of flagellins are publically available in the NCBI Genbank, see for example Accession Nos. AAL20871, NP_310689, BAB58984, AAO85383, AAA27090, NP_461698, AAK58560, YP_001217666, YP_002151351, YP_001250079, AAA99807, CAL35450, AAN74969, and BAC44986. The flagellin sequences from these and other species are intended to be encompassed by the term flagellin as used herein. Therefore, the sequence differences between species are included within the meaning of the term.

The term “flagellin polypeptide” is intended to a flagellin or a fragment thereof that retains the ability to bind and activate TLR5. As used herein the term “toll-like receptor 5” or “TLR5” has its general meaning in the art and is intended to mean a toll-like receptor 5 of any species, but preferably a human toll-like receptor 5. Upon activation, a TLR5 induces a cellular response by transducing an intracellular signal that is propagated through a series of signaling molecules from the cell surface to the nucleus. Typically, the intracellular domain of TLR5 recruits the adaptor protein, MyD88, which recruits the serine/threonine kinases IRAK (IRAK-1 and IRAK-4). IRAKs form a complex with TRAF6, which then interacts with various molecules that participate in transducing the TLR signal. These molecules and other TLR5 signal transduction pathway components stimulate the activity of transcription factors, such as fos, jun and NF-kB, and the corresponding induction of gene products of fos-, jun- and NF-kB-regulated genes, such as, for example, IL-6, TNF-alpha, CXCL1, CXCL2 and CCL20. Typically, the flagellin polypeptide of the present invention comprises the domains of flagellin involved in TLR5 signaling. The term “domain of flagellin” includes naturally occurring domain of flagellin and function conservative variants thereof. “Function conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence identity between any two proteins of similar function may vary and may be, for example, from 70% to 99%. Thus a “function-conservative variant” also includes a polypeptide which has at least 70% amino acid identity with the native sequence of flagellin or fragment thereof. According to the invention a first amino acid sequence having at least 70% of identity with a second amino acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; or 99, or 100% of identity with the second amino acid sequence. In the same manner a first amino acid sequence having at least 90% of identity with a second amino acid sequence means that the first sequence has 90; 91; 92; 93; 94; 95; 96; 97; 98; or 99, or 100% of identity with the second amino acid sequence. Amino acid sequence identity is preferably determined using a suitable sequence alignment algorithm and default parameters, such as BLAST P (Karlin and Altschul, 1990). The domains of flagellin that are involved in TLR5 signaling are well known in the art, see for example Smith et al. (2003) Nat. Immunol. 4: 1247-1253 (e.g., amino acids 78-129, 135-173 and 394-444 of S. typhimurium flagellin or homologs or modified forms thereof).

Examples of flagellin polypeptides include but are not limited to those described in U.S. Pat. Nos. 6,585,980; 6,130,082; 5,888,810; 5,618,533; and 4,886,748; U.S. Patent Publication No. US 2003/0044429 A1; and in the International Patent Application Publications no WO 2008097016 and WO 2009156405 which are incorporated by reference. An exemplary E. coli O157:H7 flagellin is SEQD ID NO:1. An exemplary S. typhimurium flagellin is SEQ ID NO:2 or SEQ ID NO:3.

Polypeptide numbering starts at the first amino-acid after the eventual N-terminal methionine (not shown in SEQ ID No 3), which is typically excised by methionine aminopeptidase in bacteria host cells as under-mentioned.

In some embodiments, amino acid sequences having at least 70% of identity with SEQ ID NO: 1 SEQ ID NO:2 or SEQ ID NO:3 can be used as flagellin polypeptides according to the invention. In some embodiments, amino acid sequences having at least 90% of identity with SEQ ID NO: 1 SEQ ID NO:2 or SEQ ID NO:3 can be used as flagellin polypeptides according to the invention. In some embodiments, amino acid sequences having at least 70% of identity with SEQ ID NO:3 can be used as flagellin polypeptides according to the invention provided that the residues 89-96 (i.e. the residues that are involved in TLR5 detection) are not mutated (i.e. not substituted or not deleted). In some embodiments, amino acid sequences having at least 90% of identity with SEQ ID NO: 1 SEQ ID NO:2 or SEQ ID NO:3 can be used as flagellin polypeptides according to the invention provided that the residues 89-96 (i.e. the residues that are involved in TLR5 detection) are not mutated (i.e. not substituted or not deleted).

In some embodiments, the present encompasses use of the flagellin recombinant polypeptides described in the International Patent Applications no WO 2009156405, and no WO 2016/102536 which are incorporated by reference in its entirely.

In some embodiments, the flagellin polypeptide of the present invention comprises: a) a N-terminal peptide having at least 90% amino acid identity with the amino acid sequence starting from the amino acid residue located at position 1 of SEQ ID NO:3 and ending at an amino acid residue selected from the group consisting of any one of the amino acid residues located at positions 99 to 173 of SEQ ID NO:3; and b) a C-terminal peptide having at least 90% amino acid identity with the amino acid sequence starting at an amino acid residue selected from the group consisting of any one of the amino acid residues located at positions 401 to 406 of SEQ ID NO:3 and ending at the amino acid residue located at position 494 of SEQ ID NO:3, wherein: the said N-terminal peptide is directly linked to the said C-terminal peptide, or the said N-terminal peptide and the said C-terminal peptide are indirectly linked, one to the other, through a spacer chain.

In some embodiments, said N-terminal peptide is selected from the group consisting of the amino acid sequences 1-99, 1-137, 1-160 and 1-173 of SEQ ID NO:3.

In some embodiments, said C-terminal peptide is selected from the group consisting of the amino acid sequences 401-494 and 406-494 of SEQ ID NO:3.

In some embodiments, said N-terminal and C-terminal peptides consist of the amino acid sequences 1-173 and 401-494 of SEQ ID NO:3, respectively.

In some embodiments, said N-terminal and C-terminal peptides consist of the amino acid sequences 1-160 and 406-494 of SEQ ID NO:3, respectively.

In some embodiments, said N-terminal and C-terminal peptides consist of the amino acid sequences 1-137 and 406-494 of SEQ ID NO:3, respectively.

In some embodiments, said N-terminal peptide and the said C-terminal peptide are indirectly linked, one to the other, through an intermediate spacer chain consisting of a NH2-GIy-AIa-AIa-GIy-COOH (SEQ ID NO:4) peptide sequence.

In some embodiments, the asparagine amino acid residue located at position 488 of SEQ ID NO:3 is replaced by a serine.

In some embodiments, the flagellin polypeptide as above described comprises an additional methionine residue at the N-terminal end (regarding flagellin polypeptide of SEQ ID No 3).

In some embodiments, the flagellin polypeptide as above described comprises one additional methionine residue (M) and one additional lysin residue (L) at the N-terminal end (amino acid residues ML) (regarding flagellin polypeptide of SEQ ID No 3).

Accordingly an example of the flagellin polypeptide corresponding to a modified recombinant flagellin (FLAMOD: see SEQ ID No 5), wherein N-terminal and C-terminal peptides consist of the amino acid sequences 1-173 and 401-494 of SEQ ID NO:3, said N-terminal peptide and the said C-terminal peptide are indirectly linked, one to the other, through an intermediate spacer chain consisting of a NH2-GIy-AIa-AIa-GIy-COOH (SEQ ID NO:4) peptide sequence and wherein said polypeptide comprises one additional methionine residue (M) and one additional lysine residue (L) at the N-terminal end.

In one embodiment, the flagellin polypeptide is the recombinant polypeptide having the amino-acid sequence of SEQ ID No 5.

The flagellin polypeptide of the present invention is produced by any method well known in the art. In some embodiments, the flagellin polypeptide of the present invention is typically recombinantly produced by recombinant cells that have been transfected with a nucleic acid that encodes its amino acid sequence and allows its effective production within the transfected cells. The nucleic acid sequence encoding the flagellin polypeptide of the invention, may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence if the sequence is to be secreted, an origin of replication, one or more marker genes, an enhancer element, a promoter, and a transcription termination sequence. Construction of suitable vectors containing one or more of these components employs standard ligation techniques that are known to the skilled artisan. Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. An example of suitable selectable markers for mammalian cells are those that enable the identification of cells competent to take up the nucleic acid encoding the flagellin polypeptide of the invention such as DHFR or thymidine kinase. An appropriate host cell when wild-type DHFR is employed is the CHO cell line deficient in DHFR activity. Expression and cloning vectors usually contain a promoter operably linked to the nucleic acid sequence encoding the flagellin polypeptide to direct mRNA synthesis. Promoters recognized by a variety of potential host cells are well known. Promoters suitable for use with prokaryotic hosts include the beta-lactamase and lactose promoter systems, alkaline phosphatase, a tryptophan (trp) promoter system, and hybrid promoters such as the tac promoter. Promoters for use in bacterial systems also will contain a Shine-Dalgamo (S. D.) sequence operably linked to the DNA encoding the flagellin polypeptide of the invention. Host cells are transfected or transformed with expression or cloning vectors described herein for flagellin polypeptide production and cultured in conventional nutrient media modified as appropriate for inducing promoters, selecting transformants, or amplifying the genes encoding the desired sequences. The culture conditions, such as media, temperature, pH, and the like, can be selected by the skilled artisan without undue experimentation. In general, principles, protocols, and practical techniques for maximizing the productivity of cell cultures can be found in Mammalian Cell Biotechnology: A Practical Approach, M. Butler, ed. (IRL Press, 1991). Suitable host cells for cloning or expressing the DNA in the vectors herein include prokaryote, yeast, or higher eukaryote cells. Suitable prokaryotes include, but are not limited to, eubacteria, such as Gram-negative or Gram-positive organisms, for example, Enterobacteriaceae such as E. coli. Various E. coli strains are publicly available, such as E. coli K12 strain MM294 (ATCC 31, 446); E. coli X1776 (ATCC 31,537); E. coli strain W3110 (ATCC 27,325); and K5772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41 P disclosed in DD 266,710 published 12 Apr. 1989), Pseudomonas such as P. aeruginosa, and Streptomyces. These examples are illustrative rather than limiting. Strain SIN41 of Salmonella typhimurium (fliC fljB), is particularly interesting for the production of flagellin polypeptides of the invention, since these prokaryotic host cells do not secrete any flagellins (Proc Natl Acad Sci USA. 2001; 98:13722-7). However flagellins are secreted through specialized secretion system: the so called “Type III secretion system”. Interestingly, strain SIN41 produces all components of the type III secretion system required for optimal flagellin secretion. Cloning sequence coding new flagellin peptides under fliC promoter enables secretion in large amounts of the flagellin polypeptides of interest in strain SIN41. Strain W3110 is also interesting because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W31 10 strain 27C7 (ATCC 55,244), which has the complete genotype tonA ptr3 phoA E15 (argF-lac)169 degP ompT kan.sup.r; E. coli W3110 strain 37D6, which has the complete genotype tona ptr3 phoA E15 (argF-lac)169 degP ompT rbs7 ilvG kan.sup.r; E. coli W31 10 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. The E. coli strains MG1655, MG1655 AfimA-H or MKS12, a fliD- and -f/m>A-/-/-deleted MG1655 strain are also interesting candidates for production of recombinant flagellins as secreted proteins (Nat Biotechnol. 2005; (4):475-81). Alternatively, in vitro methods of cloning, e.g., PCR or other nucleic acid polymerase reactions, are suitable. Flagellin polypeptide of the invention may be recovered from culture medium or from host cell lysates. If membrane-bound, it can be released from the membrane using a suitable detergent solution (e.g., TRITON-XTM. 100) or by enzymatic cleavage. In some embodiments, the flagellin polypeptide is purified from the supernatant of recombinant S. Typhimurium SIN41 (fliC fljB), as disclosed in Nempont et al. (Nempont, C. C., D.; Rumbo, M.; Bompard, C.; Villeret, V.; Sirard, J. C. 2008. Deletion of flagellin's hypervariable region abrogates antibody-mediated neutralization and systemic activation of TLR5-dependent immunity. J Immunol 181:2036-2043.). In particular, Salmonella were grown in Luria-Bertani (LB) broth for 6-18 hours at 37° C. with agitation. The supernatant was filtered and saturated with 60% ammonium sulfate (Sigma Aldrich, USA). The precipitated materials were recovered by centrifugation, solubilization in 20 mM Tris/HCl pH7.5 and then dialysis. The proteins were further purified by successive rounds of hydroxyapatite, anion exchange, and size exclusion chromatography (Bio-Rad Laboratories, USA; GE Healthcare, Sweden). Lastly, the proteins were depleted of lipopolysaccharide (LPS) using a polymyxin B column (Pierce, USA). Using the Limulus assay (Associates of Cape Cod Inc., USA), the residual LPS concentration was determined to be less than 30 pg LPS per μg recombinant flagellin. Constructs encoding the flagellins may be generated by PCR and cloned into the expression vector pET22b+. The plasmids can be introduced in Escherichia coli BL21(DE3) and protein production can be induced by adding IPTG 1 mM. After disruption on French press, the soluble fraction was depleted of lipopolysaccharide (LPS) using Triton X-114 extraction. If flagellins are found in the insoluble fraction after the French-press, inclusion bodies are denatured in presence of Urea 8M followed by dialysis and Triton X-114 extraction. The proteins can then be purified on anion exchange chromatography and gel filtration. Finally, proteins can be again depleted of LPS using a polymyxin B column (Pierce, USA).

### Buffering Agent

The term Buffering agent means a chemical that keeps the pH of a substance constant. The buffer system consists of an acid base couple which limits the pH variations in a liquid formulation.

In one embodiment, in which the buffering agent is acetate and the concentration of acetate in the liquid formulation is in the range of from about 1 mM to about 200 mM, e.g., from about 5 mM to about 150 mM or from about 5 mM to about 100 mM or from about 5 mM to about 50 mM. In one embodiment, the concentration of the acetate in the liquid formulation is in the range of from about 5 mM to about 25 mM, e.g., from about 5 mM to about 20 mM or from about 5 mM to about 15 mM or from about 7.5 mM to about 12.5 mM. In one embodiment, the concentration of the acetate in the liquid formulation is about 10 mM.

In one embodiment, in which the buffering agent is phosphate the concentration of the phosphate in the liquid formulation is in the range of from about 1 mM to about 200 mM, e.g., from about 5 mM to about 150 mM or from about 5 mM to about 100 mM or from about 5 mM to about 50 mM. In one embodiment, the concentration of the phosphate in the liquid formulation is in the range of from about 5 mM to about 25 mM, e.g., from about 5 mM to about 20 mM or from about 5 mM to about 15 mM or from about 7.5 mM to about 12.5 mM. In one embodiment, the concentration of the phosphate in the liquid formulation is about 10 mM. In one embodiment, the concentration of the phosphate in the liquid formulation is about 20 mM.

In one embodiment, the acetate acting as buffering agent is sodium acetate (or another suitable acetate salt, e.g., potassium acetate), e.g., in conjunction with acetic acid (i.e., in the form of an acetate buffer). Methods to prepare a suitable acetate buffer are well known to a person skilled in the art.

Examples of pharmaceutical grade buffer agent acetate are:
Sodium Acetate Trihydrate (CH₃COONa⁺.3H₂O)
Acetic Acid (CH₃COOH)

In one embodiment, the phosphate acting as buffering agent is phosphate sodium (or another suitable phosphate salt), e.g., in the form of a phosphate buffer. Methods to prepare a suitable phosphate buffer are well known to a person skilled in the art.

Examples of pharmaceutical grade buffer agent phosphate are:
Dibasic phosphate, anhydrous Na₂HPO₄
Monobasic phosphate, anhydrous NaH₂PO₄

In one embodiment, the liquid formulation does not comprise citrate and histidine as buffering agent.

The term “aqueous medium” (or “aqueous solution”), as used herein, refers to a liquid medium or solution in which water is the solvent. In one embodiment, the aqueous medium is/consists of water, in particular purified water or water for injection (WFI). In one embodiment, the aqueous medium is sterile. In one embodiment, the liquid formulation is sterile.

In one embodiment, the liquid formulation has a pH in the range of from about 3.5 to about 8.

In one embodiment, the buffering agent is acetate, and the liquid formulation has a pH which is lower than about 5.8 or lower than about 5.5. In one embodiment, the buffering agent is acetate, and the liquid formulation has a pH which is in the range of between about 3.5 and lower than about 5.8 or of between about 4.0 and about 5.8 or of between about 4.5 and about 5.8 or of between about 4.5 and about 5.5. In one embodiment, the liquid formulation has a pH of about 5.5.

In one embodiment, the buffering agent is acetate at a concentration of 10 mM, and the liquid formulation has a pH which is lower than about 5.8 or lower than about 5.5. In one embodiment, the buffering agent is acetate at a concentration of 10 mM, and the liquid formulation has a pH which is in the range of between about 3.5 and lower than about 5.8 or of between about 4.0 and about 5.8 or of between about 4.5 and about 5.8 or of between about 4.5 and about 5.5. In one embodiment, the liquid formulation has a pH of about 5.5 and the buffering agent is acetate at a concentration of 10 mM.

In one embodiment, the buffering agent is phosphate, and the liquid formulation has a pH which is equal to or lower than about 8 or lower than about 6.5. In one embodiment, the buffering agent is phosphate, and the liquid formulation has a pH which is lower than about 7.5 or lower than about 7. In one embodiment, the buffering agent is phosphate, and the liquid formulation has a pH which is in the range of between about 8.0 and about 6.2 or of between about 7.5 and about 6.2 or of between about 7.3 and about 6.5 or of between about 7.0 and about 6.3 or of between about 6.8 and about 6.0. In one embodiment, the liquid formulation has a pH of about 6.5.

In one embodiment, the buffering agent is phosphate at a concentration of 10 mM, and the liquid formulation has a pH which is equal to or lower than about 8 or lower than about 6.5. In one embodiment, the buffering agent is phosphate at a concentration of 10 mM, and the liquid formulation has a pH which is lower than about 7.5 or lower than about 7. In one embodiment, the buffering agent is phosphate at a concentration of 10 mM, and the liquid formulation has a pH which is in the range of between about 8.0 and about 6.2 or of between about 7.5 and about 6.2 or of between about 7.3 and about 6.5 or of between about 7.0 and about 6.3 or of between about 6.8 and about 6.0. In one embodiment, the liquid formulation has a pH of about 6.5 and the buffering agent is phosphate at a concentration of 10 mM.

The liquid formulation may comprise one or more other excipients as long as they are pharmaceutically acceptable and do not compromise the liquid formulation's suitability for administration by inhalation, in particular inhalation via a nebulizer. Suitable excipients are listed in a pharmacopoeia or in, e.g., REMINGTON's PHARMACEUTICAL SCIENCES (18th Ed., A. R. Gennaro, ed., Mack Publishing Company 1990), and subsequent editions of the same). The term “pharmaceutically acceptable”, as used herein, refers to the non-toxicity of a material, which, in one embodiment, does not interact with the action of the active agent of the liquid formulation.

### Surfactant

The liquid formulation according to the invention also comprises a surfactant consisting of polysorbate.

The term “surfactant” (or “surface active agent”), as used herein, refers to compounds that lower the surface tension (or interfacial tension) between two liquids, between a gas and a liquid, or between a liquid and a solid. The compounds lower the surface tension (or interfacial tension) between a gas (e.g., air) and a liquid. The surfactant is a non-ionic surfactant. More precisely, the surfactant consisting of polysorbate. In one embodiment polysorbate is selected from the group consisting polysorbate 20 or polysorbate 80.

In one embodiment, the surfactant is polysorbate 80 (PS80).

In one embodiment, the concentration of the polysorbate in the liquid formulation is equal to or lower than about 0.1% (w/v) or equal to or lower than about 0.05% (w/v), e.g., equal to or lower than about 0.04% (w/v) or equal to or lower than about 0.03% (w/v) or equal to or lower than about 0.02% (w/v) or equal to or lower than about 0.01% (w/v) or equal to or lower than about 0.005% (w/v). In one embodiment, the concentration of the polysorbate surfactant in the liquid formulation is lower than about 0.02% (w/v).

In one embodiment, the concentration of the PS80 in the liquid formulation is equal to or lower than about 0.1% (w/v) or equal to or lower than about 0.05% (w/v), e.g., equal to or lower than about 0.04% (w/v) or equal to or lower than about 0.03% (w/v) or equal to or lower than about 0.02% (w/v) or equal to or lower than about 0.01% (w/v) or equal to or lower than about 0.005% (w/v). In one embodiment, the concentration of PS80 in the liquid formulation is lower than about 0.02% (w/v).

In one embodiment the liquid formulation only comprises one surfactant. In one embodiment this surfactant is PS80.

Generally, an aerosol is a suspension of fine solid particles or liquid droplets, in air or another gas. According to the present invention, the term “aerosol” or “aerosol composition” refers to a suspension of droplets of the liquid formulation as defined above in a gas, e.g., air.

In one embodiment the droplets have an average diameter below 5 μm. In one embodiment the droplets have an average diameter below 4.5 μm. In one embodiment the droplets have an average diameter below 4.0 μm. In one embodiment the droplets have an average diameter below 3.5 μm.

In one embodiment, the droplets have an average diameter in the range of from about 0.5 μm to about 5 μm. In one embodiment, the droplets have an average diameter in the range of from about 0.5 μm to about 4.5 μm. In one embodiment, the droplets have an average diameter in the range of from about 0.5 μm to about 4 μm. In one embodiment, the droplets have an average diameter in the range of from about 0.5 μm to about 3.5 μm. In one embodiment, the average diameter is the volume median diameter (VMD; also referred to as Dv50 value). In one embodiment, the VMD is determined by laser diffraction, e.g., as described in U.S. Pharmacopeia (USP) 429. Droplet size can also be measured by for example interferometric laser imaging. Results may vary upon the measurement method used.

In one embodiment the liquid formulation comprises acetate buffer as buffering agent, has a pH of about 5.8 or lower than about 5.5 and comprises droplets which have an average diameter below 5.0 μm. In one embodiment the liquid formulation comprises acetate buffer as buffering agent, has a pH of about 5.8 or lower than about 5.5 and comprises droplets which have an average diameter in the range of from about 0.5 μm to about 5 μm. In one embodiment the liquid formulation comprises acetate buffer as buffering agent, has a pH in the range of between about 3.5 and lower than about 5.8 and comprises droplets which have an average diameter below 5 μm. In one embodiment the liquid formulation comprises acetate buffer as buffering agent, has a pH in the range of between about 3.5 and lower than about 5.8 and comprises droplets which have an average diameter in the range of from about 0.5 μm to about 5 μm. In one embodiment, the liquid formulation comprises acetate buffer as buffering agent, has a pH which is or of between about 3.5 and about 5.8 and comprises droplets which have an average diameter below 5 μm. In one embodiment, the liquid formulation comprises acetate buffer as buffering agent, has a pH which is or of between about 3.5 and about 5.8 and comprises droplets which have an average diameter in the range of from about 0.5 μm to about 5 μm. In one embodiment, the liquid formulation comprises acetate buffer as buffering agent, has a pH which is or of between about 4.0 and about 5.8 and comprises droplets which have an average diameter below 5 μm. In one embodiment, the liquid formulation comprises acetate buffer as buffering agent, has a pH which is or of between about 4.0 and about 5.8 and comprises droplets which have an average diameter in the range of from about 0.5 μm to about 5 μm. In one embodiment, the liquid formulation comprises acetate buffer as buffering agent, has a pH which is or of between about 4.5 and about 5.8 and comprises droplets which have an average diameter below 5 μm. In one embodiment, the liquid formulation comprises acetate buffer as buffering agent, has a pH which is or of between about 4.5 and about 5.8 and comprises droplets which have an average diameter in the range of from about 0.5 μm to about 5 μm. In another embodiment the liquid formulation only comprises one surfactant. In one embodiment this surfactant is PS80.

In one embodiment the liquid formulation comprises phosphate buffer as buffering agent, has a pH lower than about 8.0 or lower than about 6.5 and comprises droplets which have an average diameter below 5 μm. In one embodiment the liquid formulation comprises phosphate buffer as buffering agent, which is in the range of between about 8.0 and about 6.2 and comprises droplets which have an average diameter in the range of from about 0.5 μm to about 5 μm. In one embodiment the liquid formulation comprises phosphate buffer as buffering agent, has a pH in the range of between about 7.5 and lower than about 7 and comprises droplets which have an average diameter below 5 μm. In one embodiment the liquid formulation comprises phosphate buffer as buffering agent, has a pH in the range of between about 7.5 and lower than about 7 and comprises droplets which have an average diameter in the range of from about 0.5 μm to about 5 μm. In one embodiment, the liquid formulation comprises phosphate buffer as buffering agent, has a pH which is or of between about 7.5 and about 6.2 and comprises droplets which have an average diameter below 5 μm. In one embodiment, the liquid formulation comprises phosphate buffer as buffering agent, has a pH which is or of between about 7.5 and about 6.2 and comprises droplets which have an average diameter in the range of from about 0.5 μm to about 5 μm. In one embodiment, the liquid formulation comprises phosphate buffer as buffering agent, has a pH which is or of between about 7.3 and about 6.5 and comprises droplets which have an average diameter below 5 μm. In one embodiment, the liquid formulation comprises phosphate buffer as buffering agent, has a pH which is or of between about 7.3 and about 6.5 and comprises droplets which have an average diameter in the range of from about 0.5 μm to about 5 μm. In one embodiment, the liquid formulation comprises phosphate buffer as buffering agent, has a pH which is or of between about 7.0 and about 6.3 and comprises droplets which have an average diameter below 5 μm. In one embodiment, the liquid formulation comprises phosphate buffer as buffering agent, has a pH which is or of between about 7.0 and about 6.3 and comprises droplets which have an average diameter in the range of from about 0.5 μm to about 5 μm. In another embodiment the liquid formulation only comprises one surfactant. In one embodiment this surfactant is PS80.

According to the present invention, the flagellin polypeptide which are present in the aerosols or liquid formulations described herein are characterized by low aggregation, e.g., as compared to the same flagellin polypeptide being formulated in a formulation comprising citrate or histidine.

In one embodiment, the flagellin polypeptide which are present in the aerosols or liquid formulations described herein have one or more of the following properties:
the polydispersity index (PDI) of the flagellin polypeptide which are present in the aerosols or liquid formulations described herein is 0.4 or less, or 0.3 or less, or 0.2 or less, or 0.15 or less, or 0.1 or less, e.g., as determined by DLS (e.g., essentially as described in Example section);
the percentage of polydispersity of the monomers of the flagellin polypeptide which are present in the aerosols or liquid formulations described herein is 30% or less, or 25% or less, or 20% or less, or 15% or less, e.g., as determined by DLS (e.g., essentially as described in Example section);
the mass percentage of the monomers of the flagellin polypeptide which are present in the aerosols or liquid formulations described herein is 99.7% or more, or 99.8% or more, or more than 99.9%, e.g., as determined by DLS (e.g., essentially as described in Example section);
the number of particles total is less than 50000 per mL, or less than 30000 per mL, or less than 10000 per mL, or less than 5000 per mL; and
the number of particles >2 μm is less than 4000 per mL, or less than 3000 per mL, or less than 2000 per mL, or less than 1000 per mL; and
the number of particles >10 μm is less than 250 per mL, or less than 150 per mL, or less than 100 per mL; and
the number of particles >25 μm is less than 100 per mL, or less than 50 per mL, or less than 40 per mL, or less than 30 per mL,
e.g., as determined by FCM (e.g., essentially as described in Example Section).

### Method of Preparing an Aerosol Composition

In another aspect, the present invention relates to a method of preparing an aerosol composition comprising droplets comprising a liquid formulation, said method comprising the steps:
(i) providing a liquid formulation as defined above,
(ii) nebulizing the liquid formulation provided in step (i) by means of a nebulizer, thereby preparing the aerosol.

In one embodiment, the nebulizer is a mesh nebulizer.

Nebulizers allow the dispersion of a liquid in a gas to aerosolize a liquid formulation into an aerosol that is inhaled into a subject's respiratory tract. Examples of nebulizers include a soft mist nebulizer, a mesh nebulizer (e.g., a vibrating mesh nebulizer), a jet nebulizer and an ultrasonic wave nebulizer. Suitable nebulizer devices include the Aerogeno® Solo (Aerogen), Pari eFlow® (Pari GmbH), Philips I-Neb™ (Philips), the Pari LC Sprint (Pari GmbH), the AERxRTM Pulmonary Delivery System (Aradigm Corp.) and the Pari LC Plus Reusable Nebulizer (Pari GmbH). In one embodiment, the nebulizer is a mesh nebulizer, in particular a vibrating mesh nebulizer. A nebulizer typically comprises from about 1 mL to about 200 mL, more typically from 1 mL to 20 mL of the liquid formulation.

In one embodiment, the method further comprises, between steps (i) and (ii), the steps of:
(ia) lyophilizing the liquid formulation provided in step (i), thereby providing a lyophilized powder, and
(ib) reconstituting the liquid formulation provided in step (i) by adding an appropriate amount of an aqueous medium to the lyophilized powder provided in step (ia).

In another aspect, the present invention relates to an aerosol composition comprising droplets comprising a liquid formulation, wherein the aerosol is obtainable by the method as defined above. In one embodiment, the droplets have an average diameter in the range of from about 0.5 μm to about 5 μm or from about 0.5 μm to about 3.5 μm.

### Method of Delivering the Flagellin Polypeptide

In another aspect, the present invention relates to the liquid formulation as defined above or the aerosol composition as defined above for use in a method of delivering a flagellin polypeptide to the lungs of a subject, wherein the aerosol is administered to the subject by inhalation or the liquid formulation is administered to the subject by inhalation via a nebulizer.

In one embodiment, the flagellin polypeptide comprises: a) a N-terminal peptide having at least 90% amino acid identity with the amino acid sequence starting from the amino acid residue located at position 1 of SEQ ID NO:3 and ending at an amino acid residue selected from the group consisting of any one of the amino acid residues located at positions 99 to 173 of SEQ ID NO:3; and b) a C-terminal peptide having at least 90% amino acid identity with the amino acid sequence starting at an amino acid residue selected from the group consisting of any one of the amino acid residues located at positions 401 to 406 of SEQ ID NO:3 and ending at the amino acid residue located at position 494 of SEQ ID NO:3, wherein: the said N-terminal peptide is directly linked to the said C-terminal peptide, or the said N-terminal peptide and the said C-terminal peptide are indirectly linked, one to the other, through a spacer chain.

In some embodiments, said N-terminal and C-terminal peptides consist of the amino acid sequences 1-173 and 401-494 of SEQ ID NO:3, respectively.

In some embodiments, said N-terminal peptide and the said C-terminal peptide are indirectly linked, one to the other, through an intermediate spacer chain consisting of a NH2-GIy-AIa-AIa-GIy-COOH (SEQ ID NO:4) peptide sequence.

In some embodiments, the flagellin polypeptide as above described comprises one additional methionine residue (M) and one additional lysin residue (L) at the N-terminal end (amino acid residues ML) (regarding flagellin polypeptide of SEQ ID No 3).

In one embodiment, the flagellin polypeptide is the recombinant polypeptide having the amino acid sequence of SEQ ID No 5.

In one embodiment, the nebulizer is a mesh nebulizer.

The term “subject” means according to the invention a subject for treatment, in particular a diseased subject (also referred to as “patient”), including human beings, non-human primates or other animals, in particular mammals, such as cows, horses, pigs, sheep, goats, dogs, cats, rabbits or rodents, such as mice, rats, guinea pigs and hamsters. In one embodiment, the subject/patient is a human being.

In another aspect, the present invention relates to the aerosol composition of the invention or the liquid formulation as defined above optionally in combination with at least one antibiotic for use in a method of treating or preventing a lung disease in a subject, wherein the aerosol is administered to the subject by inhalation or the liquid formulation is administered to the subject by inhalation via a nebulizer.

In one embodiment, the lung disease is a lung infectious disease (ie lung bacterial infection).

In one embodiment, the nebulizer is a mesh nebulizer.

The term “lung infectious disease” (also referred to as “pulmonary infectious disease” herein) refers to any disease which can be transmitted from individual to individual or from organism to organism, and is caused by a microbial agent (e.g. common cold) that affect the lungs of a subject. Examples of infectious diseases include viral infectious diseases, such as, influenza viruses, respiratory syncytial virus (RSV), and severe acute respiratory syndrome (SARS), bacterial infectious diseases, such as Legionnaire's disease (Legionella), tuberculosis, infections by E. coli, Staphylococci, Salmonella or Streptococci (tetanus); Parasitic infectious disease such as Respiratory cryptosporidiosis or fungal infections, which are caused, e.g., by Aspergillosis.

The lung infectious disease may also be a pneumonia

The term “pneumonia” (also referred to as “Lower respiratory tract infection (LRTI)” herein) can also be applied to other types of infection including lung abscess and acute bronchitis. Symptoms include shortness of breath, weakness, fever, coughing and fatigue. A routine chest X-ray is not always necessary for people who have symptoms of a lower respiratory tract infection. Influenza affects both the upper and lower respiratory tracts. Antibiotics are the first line treatment for pneumonia; however, they are neither effective nor indicated for parasitic or viral infections. Acute bronchitis typically resolves on its own with time.

The most common cause of pneumonia is pneumococcal bacteria, Streptococcus pneumoniae accounts for 2/3 of bacteremic pneumonias. This is a dangerous type of lung infection with a mortality rate of around 25%. For optimal management of a pneumonia patient, the following must be assessed: pneumonia severity (including treatment location, e.g., home, hospital or intensive care), identification of causative organism, analgesia of chest pain, the need for supplemental oxygen, physiotherapy, hydration, bronchodilators and possible complications of emphysema or lung abscess.

The main cause of pneumonia is due to typical bacterial Infections (Haemophilus influenzae, Staphylococcus aureus, Klebsiella pneumoniae,) and atypical bacterial Infections (Legionella pneumophila, Mycoplasma pneumoniae, Chlamydophila pneumoniae, Chlamydia psittaci), Parasitic infections (Respiratory cryptosporidiosis) and viral infections (Adenovirus, Influenza A virus, Influenza B virus, Human parainfluenza viruses, Human respiratory syncytial virus, Severe acute respiratory syndrome coronavirus (SARS-CoV), Middle East respiratory syndrome coronavirus (MERS-CoV), Severe acute respiratory syndrome coronavirus 2 (SARS-CoV-2))

In another aspect, the present invention relates to a method of delivering a flagellin polypeptide to the lungs of a subject, said method comprising administering to the subject an effective amount of the aerosol as defined above by inhalation or administering to the subject an effective amount of the liquid formulation as defined above by inhalation via a nebulizer.

In one embodiment, the flagellin polypeptide comprises: a) a N-terminal peptide having at least 90% amino acid identity with the amino acid sequence starting from the amino acid residue located at position 1 of SEQ ID NO:3 and ending at an amino acid residue selected from the group consisting of any one of the amino acid residues located at positions 99 to 173 of SEQ ID NO:3; and b) a C-terminal peptide having at least 90% amino acid identity with the amino acid sequence starting at an amino acid residue selected from the group consisting of any one of the amino acid residues located at positions 401 to 406 of SEQ ID NO:3 and ending at the amino acid residue located at position 494 of SEQ ID NO:3, wherein: the said N-terminal peptide is directly linked to the said C-terminal peptide, or the said N-terminal peptide and the said C-terminal peptide are indirectly linked, one to the other, through a spacer chain.

In some embodiments, said N-terminal and C-terminal peptides consist of the amino acid sequences 1-173 and 401-494 of SEQ ID NO:3, respectively.

In some embodiments, said N-terminal peptide and the said C-terminal peptide are indirectly linked, one to the other, through an intermediate spacer chain consisting of a NH2-GIy-AIa-AIa-GIy-COOH (SEQ ID NO:4) peptide sequence.

In some embodiments, the flagellin polypeptide as above described comprises one additional methionine residue (M) and one additional lysin residue (L) at the N-terminal end (amino acid residues ML) (regarding flagellin polypeptide of SEQ ID No 3).

In one embodiment, the flagellin polypeptide is the recombinant polypeptide having the amino acid sequence of SEQ ID No 5.

In one embodiment, the nebulizer is a mesh nebulizer.

The term “effective amount”, as used herein, refers, in particular, to a “therapeutically effective amount”, which is an amount that achieves a desired therapeutic reaction or a desired therapeutic effect alone or together with further doses, particularly without causing unacceptable side-effects. In the case of treatment of a particular disease or of a particular condition, the desired reaction particularly relates to inhibition of the course of the disease. This comprises slowing down the progress of the disease and, in particular, interrupting or reversing the progress of the disease. The desired reaction in a treatment of a disease or of a condition may also be delay of the onset or a prevention of the onset of said disease or said condition. An effective amount of an aerosol or liquid formulation as described herein, and, thus, of the flagellin polypeptide contained therein, will depend on the condition to be treated, the severity of the disease, the individual parameters of the subject, including age, physiological condition, size and weight, the duration of treatment, the type of an accompanying therapy (if present), the specific route of administration and similar factors. Accordingly, the doses administered of the aerosol or liquid formulation described herein may depend on several of such parameters. In the case that a reaction in a subject is insufficient with an initial dose, higher doses may be used.

In another aspect, the present invention relates to a method of treating or preventing a lung infectious disease in a subject, said method comprising administering to the subject an effective amount of the aerosol as defined above by inhalation or administering to the subject an effective amount of the liquid formulation as defined above optionally in combination with at least one antibiotic by inhalation via a nebulizer.

In one embodiment, the lung infectious disease is a lung bacterial infection.

In one embodiment, the nebulizer is a mesh nebulizer.

In another aspect, the present invention relates to a nebulizer comprising a liquid formulation as defined above.

In one embodiment, the nebulizer is a mesh nebulizer.

### Kit

In another aspect, the present invention relates to a kit comprising
(i) a container comprising the liquid formulation as defined above or a powder obtainable by lyophilization of the liquid formulation, and
   (ii) a nebulizer.

In one embodiment, the nebulizer is a mesh nebulizer.

As used herein, the term “kit of parts (in short: kit)” refers to an article of manufacture comprising one or more containers, a nebulizer (e.g., a mesh nebulizer), and, optionally, a data carrier. Said one or more containers are filled with the liquid formulation as defined above and/or a powder obtainable by lyophilization of the liquid formulation. Additional containers may be included in the kit that contain, e.g., diluents (e.g., an aqueous medium), buffers and further reagents as defined herein. Said data carrier may be a non-electronical data carrier, e.g., a graphical data carrier such as an information leaflet, an information sheet, a bar code or an access code, or an electronical data carrier such as a compact disk (CD), a digital versatile disk (DVD), a microchip or another semiconductor-based electronical data carrier. The access code may allow the access to a database, e.g., an internet database, a centralized, or a decentralized database. Said data carrier may comprise instructions for the use of the kit in the methods and uses as described herein.

### Use of the Liquid Formulations

In another aspect, the present invention relates to the use of a liquid formulation as defined above for preparing an aerosol by nebulization by means of a nebulizer.

In one embodiment, the nebulizer is a mesh nebulizer.

In another aspect, the present invention relates to the use of a buffering agent selected from the group consisting of acetate, phosphate and combinations thereof and a surfactant consisting of polysorbate for increasing the stability of a flagellin polypeptide upon nebulization of a liquid formulation comprising the flagellin polypeptide by means of a nebulizer, wherein the buffering agent is included in the liquid formulation prior to nebulization.

In one embodiment, the flagellin polypeptide comprises: a) a N-terminal peptide having at least 90% amino acid identity with the amino acid sequence starting from the amino acid residue located at position 1 of SEQ ID NO:3 and ending at an amino acid residue selected from the group consisting of any one of the amino acid residues located at positions 99 to 173 of SEQ ID NO:3; and b) a C-terminal peptide having at least 90% amino acid identity with the amino acid sequence starting at an amino acid residue selected from the group consisting of any one of the amino acid residues located at positions 401 to 406 of SEQ ID NO:3 and ending at the amino acid residue located at position 494 of SEQ ID NO:3, wherein: the said N-terminal peptide is directly linked to the said C-terminal peptide, or the said N-terminal peptide and the said C-terminal peptide are indirectly linked, one to the other, through a spacer chain.

In some embodiments, said N-terminal and C-terminal peptides consist of the amino acid sequences 1-173 and 401-494 of SEQ ID NO:3, respectively.

In some embodiments, said N-terminal peptide and the said C-terminal peptide are indirectly linked, one to the other, through an intermediate spacer chain consisting of a NH2-GIy-AIa-AIa-GIy-COOH (SEQ ID NO:4) peptide sequence.

In some embodiments, the flagellin polypeptide as above described comprises one additional methionine residue (M) and one additional lysin residue (L) at the N-terminal end (amino acid residues ML) (regarding flagellin polypeptide of SEQ ID No 3).

In one embodiment, the flagellin polypeptide is the polypeptide having the amino acid sequence of SEQ ID No 5.

In one embodiment, the nebulizer is a mesh nebulizer.

In one embodiment, the term “increasing the stability” refers to preventing or reducing the extent of aggregation of the flagellin polypeptide.

In one embodiment, the biological and/or physico-chemical stability ensured by the flagellin concentration is between 10 μg/mL and 2.5 mg/mL.

In one embodiment, the liquid formulation has a pH which is equal to or lower than about 8.

In one embodiment, the liquid formulation does not comprise citrate or histidine.

In one embodiment, the liquid formulation has a pH in the range of from about 3.5 to about 8.

In one embodiment, the buffering agent is acetate, and the liquid formulation has a pH which is lower than about 5.8 or lower than about 5.5. In one embodiment, the buffering agent is acetate, and the liquid formulation has a pH which is in the range of between about 3.5 and lower than about 5.8 or of between about 4.0 and about 5.8 or of between about 4.5 and about 5.8 or of between about 4.5 and about 5.5. In one embodiment, the liquid formulation has a pH of about 5.5.

The liquid formulation further comprises a surfactant consisting of polysorbates.

In one embodiment, the surfactant is polysorbate 80.

In one embodiment, the concentration of the surfactant in the liquid formulation is equal to or lower than about 0.1% (w/v) or equal to or lower than about 0.02% (w/v) or equal to or lower than about 0.005% (w/v).

According to the Beasley study (Beasley R, Rafferty P, Holgate ST (1988) Adverse reactions to the non-drug constituents of nebuliser solutions. Br J Clin Pharmacol 25:283-287) preparations intended for inhalation must have an osmolality which must be between 150 and 549 mOsmol/Kg and it is recommended to be close to iso-osmolality (280-300 mOsmol/Kg). NaCl is generally used to adjust the osmolality of a formulation.

In one embodiment, the liquid formulation comprise NaCl.

In one embodiment, the liquid formulation comprise a non-buffering salt. The term “non-buffering salt”, as used herein, refers to a salt that does not or not substantially contribute to retaining the pH of the liquid formulation upon addition of an acid or a base. In one embodiment, the non-buffering salt is a halogen salt (e.g., comprising Cl− or Br−). In one embodiment, the non-buffering salt is a halogen salt that comprises one or more cations of sodium (Na+), potassium (K+), calcium (Ca2+) or magnesium (Mg2+). In one embodiment, the non-buffering salt is a halogen salt that comprises one or more cations of sodium (Na+) or potassium (K+). In yet another embodiment, the non-buffering salt is selected from the group consisting of NaCl, KCl, CaCl2) and MgCl2.

### Method of Treating

The present invention relates to a method of treating a lung bacterial infection in a subject in need thereof comprising administering the subject with a therapeutically effective amount the aerosol composition of the invention or the liquid formulation as defined above optionally in combination with at least one antibiotic.

The subject can be human or any other animal (e.g., birds and mammals) susceptible to lung bacterial infection (e.g. domestic animals such as cats and dogs; livestock and farm animals such as horses, cows, pigs, chickens, etc.). Typically said subject is a mammal including a non-primate (e.g., a camel, donkey, zebra, cow, pig, horse, goat, sheep, cat, dog, rat, and mouse) and a primate (e.g., a monkey, chimpanzee, and a human). In some embodiments, the subject is a human.

As used herein, the term “lung bacterial infection” has its general meaning in the art and refers to a bacterial infection (e.g. bacterial pneumonia) which occurs in a subject. The method of the present invention is particularly suitable for the treatment of a bacterial infection such as, but not limited to infections of the lower respiratory tract (e.g., pneumonia), middle ear infections (e.g., otitis media) and bacterial sinusitis. The bacterial infection may be caused by numerous bacterial pathogens. For example, they may be mediated by at least one organism selected from the group consisting of: Streptococcus pneumoniae; Staphylococcus aureus; Haemophilus influenza, Myoplasma species and Moraxella catarrhalis.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

The method of the present invention is particularly suitable for subjects who are identified as at high risk for developing a bacterial infection, including subjects who are at least 50 years old, subjects who reside in chronic care facilities, subjects who have chronic disorders of the pulmonary or cardiovascular system, subjects who required regular medical follow-up or hospitalization during the preceding year because of chronic metabolic diseases (including diabetes mellitus), renal dysfunction, hemoglobinopathies, or immunosuppression (including immunosuppression caused by medications or by human immunodeficiency [HIV] virus); children less than 14 years of age, patients between 6 months and 18 years of age who are receiving long-term aspirin therapy, and women who will be in the second or third trimester of pregnancy during the influenza season. More specifically, it is contemplated that the method of the invention is suitable for the treatment of bacterial superinfection post-influenza in subjects older than 1 year old and less than 14 years old (i.e., children); subjects between the ages of 50 and 65, and adults who are older than 65 years of age.

In some embodiments, the antibiotic is selected from the group consisting of aminoglycosides, beta lactams, quinolones or fluoroquinolones, macrolides, sulfonamides, sulfamethaxozoles, tetracyclines, streptogramins, oxazolidinones (such as linezolid), rifamycins, glycopeptides, polymixins, lipo-peptide antibiotics.

Tetracyclines belong to a class that shares a four-membered ring structure composed of four fused 6-membered (hexacyclic) rings. The tetracyclines exhibit their activity by inhibiting the binding of the aminoacyl tRNA to the 30S ribosomal subunit in susceptible bacteria. Tetracyclines for use in the invention include chlortetracycline, demeclocycline, doxycycline, minocycline, oxytetracycline, chlortetracycline, methacycline, mecocycline, tigecycline, limecycline, and tetracycline. The tetracyclines are effective against many known organisms including a-hemolytic streptococci, nonhemolytic streptococci, gram negative bacilli, rickettsiae, spirochetes, Mycoplasma, and Chlamydia.

Aminoglycosides are compounds derived from species of Streptomyces or Micomonospora bacteria and are primarily used to treat infections caused by gram-negative bacteria. Drugs belonging to this class all possess the same basic chemical structure, i.e., a central hexose or diaminohexose molecule to which two or more amino sugars are attached by a glycosidic bond. The aminoglycosides are bactericidal antibiotics that bind to the 30S ribosome and inhibit bacterial protein synthesis. They are active primarily against aerobic gram-negative bacilli and staphylococci. Aminoglycoside antibiotics for use in the invention include amikacin (Amikin®), gentamicin (Garamycin®), kanamycin (Kantrex®), neomycin (Mycifradin®), netilmicin (Netromycin®), paromomycin (Humatin®), streptomycin, and tobramycin (TOBI Solution®, TobraDex®).

Macrolides are a group of polyketide antibiotic drugs whose activity stems from the presence of a macrolide ring (a large 14-, 15-, or 16-membered lactone ring) to which one or more deoxy sugars, usually cladinose and desosamine, are attached. Macrolides are primarily bacteriostatic and bind to the 50S subunit of the ribosome, thereby inhibiting bacterial synthesis. Macrolides are active against aerobic and anaerobic gram positive cocci (with the exception of enterococci) and against gram-negative anaerobes. Macrolides for use in the invention include azithromycin (Zithromax®), clarithromycin (Biaxin®), dirithromycin (Dynabac®), erythromycin, clindamycin, josamycin, roxithromycin and lincomycin.

Ketolides belong to a class of semi-synthetic 14-membered ring macrolides in which the erythromycin macrolactone ring structure and the D-desosamine sugar attached at position 5 are retained, however, replacing the L-cladinose5 moiety and hydroxyl group at position 3 is a3-keto functional group. The ketolides bind to the 23S rRNA, and their mechanism of action is similar to that of macrolides (Zhanel, G. G., et al., Drugs, 2001; 61(4):443-98). The ketolides exhibit good activity against gram-positive aerobes and some gram-negative aerobes, and possess excellent activity against Streptococcus spp. including mefA and ermB-producing Streptococcus pneumoniae, and Haemophilus influenzae. Representative ketolides for use in the invention include telithromycin (formerly known as HMR-3647), HMR 3004, HMR 3647, cethromycin, EDP-420, and ABT-773.

Structurally, the quinolones possess a 1,4 dihydro-4-oxo-quinolinyl moiety bearing an essential carboxyl group at position 3. Functionally, the quinolones inhibit prokaryotic type II topoisomerases, namely DNA gyrase and, in a few cases, topoisomerase IV, through direct binding to the bacterial chromosome. Quinolones for use in the invention span first, second, third and fourth generation quinolones, including fluoroquinolones. Such compounds include nalidixic acid, cinoxacin, oxolinic acid, flumequine, pipemidic acid, rosoxacin, norfloxacin, lomefloxacin, ofloxacin, enrofloxacin, ciprofloxacin, enoxacin, amifloxacin, fleroxacin, gatifloxacin, gemifloxacin, clinafloxacin, sitafloxacin, pefloxacin, rufloxacin, sparfloxacin, temafloxacin, tosufloxacin, grepafloxacin, levofloxacin, moxifloxacin, and trovafloxacin. Additional quinolones suitable for use in the invention include those described in Hooper, D., and Rubinstein, E., “Quinolone Antimicrobial Agents, Vd Edition”, American Society of Microbiology Press, Washington D.C. (2004).

Drugs belonging to the sulfonamide class all possess a sulfonamide moiety, —SO2NH2, or a substituted sulfonamide moiety, where one 15 of the hydrogens on the nitrogen is replaced by an organic substituent. Illustrative N-substituents include substituted or unsubstituted thiazole, pyrimidine, isoxazole, and other functional groups. Sulfonamide antibiotics all share a common structural feature, i.e., they are all benzene sulfonamides, 20 meaning that the sulfonamide functionality is directly attached to a benzene ring. The structure of sulfonamide antibiotics is similar to p-aminobenzoic acid (PABA), a compound that is needed in bacteria as a substrate for the enzyme, dihydropteroate synthetase, for the synthesis of tetrahydro-25 folic acid. The sulfonamides function as antibiotics by interfering with the metabolic processes in bacteria that require PABA, thereby inhibiting bacterial growth and activity. Sulfonamide antibiotics for use in the invention include the following: mafenide, phtalylsulfathiazole, succinylsulfathiazole, sulfacetamide, sulfadiazine, sulfadoxine, sulfamazone, sulfamethazine, sulfamethoxazole, sulfametopirazine, sulfametoxypiridazine, sulfametrol, sulfamonomethoxine, sulfamylon, sulfanilamide, sulfaquinoxaline, sulfasalazine, sulfathiazole, sulfisoxazole, sulfisoxazole diolamine, and sulfaguanidine.

All members of beta-lactams possess a beta-lactam ring and a carboxyl group, resulting in 55 similarities in both their pharmacokinetics and mechanism of action. The majority of clinically useful beta-lactams belong to either the penicillin group or the cephalosporin group, including cefamycins and oxacephems. The beta-lactams also include the carbapenems and monobactams. Generally speaking, beta-lactams inhibit bacterial cell wall synthesis. More specifically, these antibiotics cause ‘nicks’ in the peptidoglycan net of the cell wall that allow the bacterial protoplasm to flow from its protective net into the surrounding hypotonic medium. Fluid then accumulates in the naked 65 protoplast (a cell devoid of its wall), and it eventually bursts, leading to death of the organism. Mechanistically, beta-lactarns act by inhibiting D-alanyl-D-alanine transpeptidase activity by forming stable esters with the carboxyl of the open lactam ring attached to the hydroxyl group of the enzyme target site. Beta-lactams are extremely effective and typically are of low toxicity. As a group, these drugs are active against many gram-positive, gram-negative and anaerobic organisms. Drugs falling into this category include 2-(3-alanyl)clavam, 2-hydroxymethylclavam, 7-methoxycephalosporin, epi-thienamycin, acetyl-thienamycin, amoxicillin, apalcillin, aspoxicillin, azidocillin, azlocillin, aztreonam, bacampicillin, blapenem, carbenicillin, carfecillin, carindacillin, carpetimycin A and B, cefacetril, cefaclor, cefadroxil, cefalexin, cefaloglycin, cefaloridine, cefalotin, cefamandole, cefapirin, cefatrizine, cefazedone, cefazolin, cefbuperazone, cefcapene, cefdinir, cefditoren, cefepime, cefetamet, cefixime, cefinenoxime, cefinetazole, cefminox, cefmolexin, cefodizime, cefonicid, cefoperazone, ceforamide, cefoselis, cefotaxime, cefotetan, cefotiam, cefoxitin, cefozopran, cefpiramide, cefpirome, cefpodoxime, cefprozil, cefquinome, cefradine, cefroxadine, cefsulodin, ceftazidime, cefteram, ceftezole, ceftibuten, ceftizoxime, ceftriaxone, cefuroxime, cephalosporin C, cephamycin A, cephamycin C, cephalothin, chitinovorin A, chitinovorin B, chitinovorin C, ciclacillin, clometocillin, cloxacillin, cycloserine, deoxy pluracidomycin B and C, dicloxacillin, dihydro pluracidomycin C, epicillin, epithienamycin D, E, and F, ertapenem, faropenem, flomoxef, flucloxacillin, hetacillin, imipenem, lenampicillin, loracarbef, mecillinam, meropenem, metampicillin, meticillin (also referred to as methicillin), mezlocillin, moxalactam, nafcillin, northienamycin, oxacillin, panipenem, penamecillin, penicillin G, N, and V, phenethicillin, piperacillin, povampicillin, pivcefalexin, povmecillinam, pivmecillinam, pluracidomycin B, C, and D, propicillin, sarmoxicillin, sulbactam, sultamicillin, talampicillin, temocillin, terconazole, thienamycin, and ticarcillin.

Over 400 natural antimicrobial peptides have been isolated and characterized. Based on chemical structure, these peptides may be classified into two main groups: linear and cyclic (R. E. Hancock et al, Adv. Microb. Physiol., 1995, 37: 135-137; H. Kleinkauf et al., Criti. Rev. Biotechnol., 198, 8: 1-32; D. Perlman and M. Bodansky, Annu. Rev. Biochem., 1971, 40: 449-464. The mode of action for the majority of these peptides (both linear and cyclic) is believed to involve membrane disruption, leading to cell leakage (A. Mor, Drug Develop. Res., 2000, 50: 440-447). The linear peptides, such as magainins and melitting, exist mainly as a-helical amphipathic structures (containing segregated hydrophobic and hydrophilic moieties), or as β-helices as found in gramicidin A (GA). Cyclic peptides, which mainly adopt an amphipatic β-sheet structures can be further divided into two subgroups: those containing disulfide bonds, such as tachyplesin, and those that do not, such as gramicidin S (D. Audreu and L. Rivas, Biopolymers, 1998, 47: 415-433). Peptide antibiotics also fall into two classes: non-ribosomally synthesized peptides, such as the gramicicins, polymyxins, bacitracins, glycopeptides, etc., and ribosomally synthesized (natural) peptides. The former are often drastically modified and are largely produced by bacteria, whereas the latter are produced by all species of life (including bacteria) as a major component of the natural host defense molecules of these species. In certain embodiments, the peptide antibiotic is a lipopeptide antibiotic such as colistin, daptomycin, surfactin, friulimicin, aculeacin A, iturin A, and tsushimycin. [00162] Colistin (also called Colimycin) is a polymixin antibiotic discovered more than 50 years ago. It is a cyclic lipopeptide antibiotic which penetrates the cell wall of Gram negative bacteria by self-induced mechanism by chelating divalent ions. Colistin destabilizes the wall and can insinuate into it. Colistin basically perforates the cell wall, causing distortion of this structure and the release of intracellular constituents. Increasing multidrug resistance in Gram-negative bacteria, in particular Pseudomonas aeruginosa, Acinetobacter baumannii, and Klebsiella pneumoniae, presents a critical problem. Limited therapeutic options have forced infectious disease clinicians and microbiologists to reappraise the clinical application of Colistin. Colistin is associated with neurotoxicity and nephrotoxicity. Dosage regimen and novel formulation may be an answer to address the toxicity issue.

In some embodiments, the aerosol composition or the liquid formulation comprising the flagellin polypeptide is used in combination with amoxicillin.

In some embodiments, the aerosol composition or the liquid formulation comprising flagellin polypeptide is used in combination with Bactrim® which contains both sulfamethoxazole and trimethoprime.

In some embodiments, the flagellin polypeptide and the antibiotic are to be used simultaneous or sequentially within a given time. The antibiotic can be applied in either order, e.g. the antibiotic can be applied first and then the flagellin polypeptide can be applied or vice versa. It is obvious that when a composition comprising both the antibiotic and flagellin polypeptide is used both components will be applied at the same time by the same routes or by different routes of administration. For example, the antibiotic may be administered to the subject via the oral route and the flagellin polypeptide is administered to the subject via the intravenous route or via the intranasal route.

By a “therapeutically effective amount” is meant a sufficient amount of the flagellin polypeptide and/or antibiotic for the treatment of a bacterial superinfection post influenza at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day, in particular 0.01 to 0.5 mg. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically the active ingredient of the present invention (i.e. the flagellin polypeptide and/or antibiotic) is combined with pharmaceutically acceptable excipients, and optionally sustained-release matrices, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

In some embodiments, the pharmaceutical composition of the invention is administered topically (i.e. in the respiratory tract of the subject). Therefore, the compositions can be formulated in the form of a spray, aerosol, solution, emulsion, or other form well-known to one of skill in the art. If the method of the invention comprises intranasal administration of a composition, the composition can be formulated in an aerosol form, spray, mist or in the form of drops. In particular, the active ingredients for use according to the present invention can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

The invention will be further illustrated by the following figures and examples. However, these examples and figures should not be interpreted in any way as limiting the scope of the present invention.

### EXAMPLE

### Materials & Methods

### Flagellin

Flagellin was supplied in PBS buffer at pH 7.4 at 1.2 g/L or 2.5 g/L. For the different formulations of the study, the Flagellin was reformulated by dialysis to change the buffer and then, the concentration was adjusted and excipients added when necessary. All formulations contained NaCl at 145 mM to maintain iso-osmolality.

### Analytical Methods to Evaluate the Aggregation

### Dynamic Light Scattering (DLS)

Dynamic light scattering (DLS) was used to determine the particles size distribution in the submicronic range. Measurements were performed with the DynaPro NanoStar (Wyatt Technology) instrument using a 663 nm laser wavelength. 100 μL of each sample was introduced in a disposable cuvette Uvette (Eppendorf) and the measurement was performed by 10 acquisitions of 7 seconds. The data were analyzed with Dynamics 7.9.0.5 software (Wyatt Technology)

The results are displayed as polydispersity index (PDI), Z-average, monomer radius (nm), percent of polydispersity of monomer pic (% pd), percent in mass of the monomer.

### Flow Cell Microscopy

Flow cell microscopy (FCM) was used to analyze the subvisible particles. Measurements were performed with the Flowcell FC200-IPAC (Occhio) instrument. 250 μL of each sample was introduced in a disposable cone and the analysis was done on 200 μL. The data were analyzed with the Callisto 3D software (Occhio).

The results are displayed as the concentration of total particles (particles/mL), particles >2 μm, >10 μm and >25 μm. For each formulation, the values before and after nebulization are presented. The values after nebulization were obtained by subtracting the concentration of particles generated after nebulization of Flagellin minus the concentration of particles generated after nebulization of the corresponding buffer with excipients (w/o Flagellin). In the case of negative value, the adopted value was 0.

### Aerosol Characterization

The aerosol was characterized by laser diffraction using a Spraytec instrument (Malvern) equipped with a vertical inhalation cell. The aerosol was aspirated into the inhalation cell with vacuum pump at a flow rate of approximately 30 L/min. The nebulizer filled with 1 mL of sample was placed on the top of the inhalation cell. The measurement duration was at least one minute.

The results are expressed as volume median diameter (VMD) and percentage of droplets with a diameter under 5 μm and between 0.5 and 3.0 μm.

### Activity Assay

The biological activity of flagellin was evaluated with HEK-Dual™ hTLR5 (NF/IL8) reporter cells expressing specifically the Flagellin target, the TLR5 receptor. The stimulation of the TLR5 is measured by the activation of IL-8 pathway and more precisely the detection of the interleukin 8 (IL-8)-dependent expression of luciferase. For this purpose, 5×10⁴ HEK-Dual™ hTLR5 (NF/IL8) cells were incubated with Flagellin before and after nebulization at eight different concentrations, ranging from 2×10⁻⁶ to 2×10⁻¹³ g/L, in a 96-well plate. After an incubation of 18 h at 37° C./5% CO2, 10 μL of supernatant were added in a new 96-well plate and mixed with 50 μL QuantiLUC reagent. The luciferase response was measured immediately after, using the luminometer Centro XS3 LB960 (Berthold). A dose-response curve of the relative light unit in function of the flagellin concentration was used to determine the EC₅₀ of the Flagellin.

The results are expressed as EC₅₀ in μg/mL.

### Results:

### Example 1

### Step 1: Determining the Impact of the Buffer on Flagellin Stability During Mesh-Nebulization

In this regard, nebulization stress was applied to Flagellin formulated in different buffers with and without surfactant.

Flagellin was prepared at a concentration of 0.5 g/L. Histidine pH 5.5, Citrate pH 5.5, Acetate pH 5.5 and Phosphate pH 6.5 buffers were employed. First, the buffers were tested without surfactant. In a second phase, the buffer were tested with a surfactant, the Polysorbate 80 (PS80) at a concentration of 0.1%.

1 mL of Flagellin, formulated in the different buffers, was nebulized with a vibrating mesh nebulizer, the Solo (Aerogen).

The degree of aggregation was measured using Flow Cell Microscopy (FCM) and Dynamic Light Scattering (DLS). The results are summarized in the Tables below

### Without Surfactant

| | | Particles | Particles >2 | Particles >10 | Particles >25 |
|---|---|---|---|---|---|
| | | total/mL | μm/mL | μm/mL | μm/mL |
| Histidine | Flagellin before | 14987 ± | 2533 ± | 5 ± | 0 ± |
| w/o PS80 | nebulization | 490 | 373 | 5 | 0 |
| | Flagellin after | 44344957 ± | 2614758 ± | 139 ± | 0 ± |
| | nebulization | 18057304 | 2834573 | 148 | 0 |
| Citrate | Flagellin before | 2821 ± | 465 ± | 37 ± | 3 ± |
| w/o PS80 | nebulization | 89 | 75 | 32 | 5 |
| | Flagellin after | 35419022 ± | 2876853 ± | 749 ± | 0 ± |
| | nebulization | 14859436 | 3366831 | 1281 | 4 |
| Acetate | Flagellin before | 4023 ± | 715 ± | 22 ± | 5 ± |
| w/o PS80 | nebulization | 172 | 55 | 23 | 5 |
| | Flagellin after | 38430491 ± | 4150700 ± | 1628 ± | 0 ± |
| | nebulization | 14817201 | 3883187 | 3528 | 0 |
| Phosphate | Flagellin before | 3544 ± | 1361 ± | 40 ± | 5 ± |
| w/o PS80 | nebulization | 2353 | 1074 | 28 | 5 |
| | Flagellin after | 8297880 ± | 492779 ± | 425 ± | 19 ± |
| | nebulization | 4067956 | 776329 | 634 | 25 |

Before nebulization, the number of subvisible particles is low especially for citrate and acetate. After nebulization, a high aggregation was observed by FCM as the number of total particles was greater than five millions in all buffers and more than one million of particles over 2 μm/mL was observed excepted in phosphate buffer which exhibited a less marked aggregation The DLS analysis confirmed those observation. Before nebulization, the PDI is multimodal for all buffers but the percentage of mass of the monomer was high signing a low aggregation. The DLS results also showed a high aggregation (submicronic particles) after nebulization in all buffers, as there were no more flagellin monomers and a major increase of the Z-average.

| | | Global | | Monomer | | |
|---|---|---|---|---|---|---|
| | | | Z-average | Radius | | |
| | | PDI | (nm) | (nm) | % pd | % mass |
| Histidine | Flagellin | Multimodal | 13.6 ± | 4.3 ± | 32.2 ± | 98.3 ± |
| w/o PS80 | before | | 0.2 | 0.2 | 6.7 | 0.2 |
| | nebulization | | | | | |
| | Flagellin | 0.237 ± | 367.6 ± | | | |
| | after | 0.068 | 43.5 | | | |
| | nebulization | | | | | |
| Citrate | Flagellin | Multimodal | 6.3 ± | 16.4 ± | 138.9 ± | 99.9 ± |
| w/o PS80 | before | | 0.4 | 10.9 | 81.6 | 0.1 |
| | nebulization | | | | | |
| | Flagellin | Multimodal | 402.3 ± | | | |
| | after | | 109.9 | | | |
| | nebulization | | | | | |
| Acetate | Flagellin | Multimodal | 5.6 ± | 10.6 ± | 97.4 ± | 99.9 ± |
| w/o PS80 | before | | 0.1 | 3.9 | 36.7 | 0.1 |
| | nebulization | | | | | |
| | Flagellin | 0.327 ± | 518.3 ± | | | |
| | after | 0.167 | 147 | | | |
| | nebulization | | | | | |
| Phosphate | Flagellin | Multimodal | 29.5 ± | 5.1 ± | 39.5 ± | 98.6 ± |
| w/o PS80 | before | | 0.9 | 0.5 | 8.3 | 0.4 |
| | nebulization | | | | | |
| | Flagellin | 0.208 ± | 324.2 ± | | | |
| | after | 0.07 | 19.3 | | | |
| | nebulization | | | | | |

### With Surfactant

| | | Particles | Particles >2 | Particles >10 | Particles >25 |
|---|---|---|---|---|---|
| | | total | μm/mL | μm/mL | μm/mL |
| Histidine | Flagellin before | 4316 ± | 145 ± | 16 ± | 0 ± |
| 0.1% PS80 | nebulization | 427 | 29 | 21 | 0 |
| | Flagellin after | 59366 ± | 316 ± | 11 ± | 0 ± |
| | nebulization | 67689 | 360 | 18 | 0 |
| Citrate | Flagellin before | 352 ± | 148 ± | 16 ± | 0 ± |
| 0.1% PS80 | nebulization | 136 | 71 | 21 | 0 |
| | Flagellin after | 30325 ± | 2700 ± | 151 ± | 11 ± |
| | nebulization | 15264 | 2446 | 206 | 25 |
| Acetate | Flagellin before | 4195 ± | 976 ± | 36 ± | 0 ± |
| 0.1% PS80 | nebulization | 794 | 379 | 63 | 0 |
| | Flagellin after | 3607 ± | 507 ± | 52 ± | 12 ± |
| | nebulization | 4849 | 701 | 61 | 14 |
| Phosphate | Flagellin before | 2630 ± | 729 ± | 96 ± | 27 ± |
| 0.1% PS80 | nebulization | 2393 | 851 | 125 | 46 |
| | Flagellin after | 1156 ± | 858 ± | 188 ± | 26 ± |
| | nebulization | 1595 | 1192 | 260 | 37 |

Before nebulization, the number of subvisible particles is low, especially for citrate. After nebulization, the amount of subvisible particles observed by FCM was lower in flagellin formulated with PS80, as compared to the buffer w/o PS80. Acetate and phosphate presented the lowest concentration of total particles and the number of particles >2 μm was low in all buffers, except citrate buffer.

| | | Global | | Monomer | | |
|---|---|---|---|---|---|---|
| | | | Z-average | Radius | | |
| | | PDI | (nm) | (nm) | % pd | % mass |
| Histidine | Flagellin | NA | | | | |
| 0.1% | before | | | | | |
| PS80 | nebulization | | | | | |
| | Flagellin after | Multimodal | 33.4 ± | 5.2 ± | 25 ± | 95.4 ± |
| | nebulization | | 6.2 | 0.6 | 7.1 | 3.7 |
| Citrate | Flagellin | 0.195 ± | 5.4 ± | 5.6 ± | 30.4 ± | 100 ± |
| 0.1% | before | 0.012 | 0.1 | 0.4 | 9.6 | 0 |
| PS80 | nebulization | | | | | |
| | Flagellin after | Multimodal | 6.9 ± | 5.5 ± | 27.7 ± | 99.8 ± |
| | nebulization | | 1.6 | 0.2 | 4.2 | 0.1 |
| Acetate | Flagellin | 0.119 ± | 5.1 ± | 5.3 ± | 23.7 ± | 99.9 ± |
| 0.1% | before | 0.022 | 0.1 | 0.1 | 1.1 | 0.1 |
| PS80 | nebulization | | | | | |
| | Flagellin after | 0.314 ± | 5.6 ± | 5.3 ± | 23.9 ± | 99.9 ± |
| | nebulization | 0.084 | 0.3 | 0.1 | 2.6 | 0.1 |
| Phosphate | Flagellin | Multimodal | 11.6 ± | 5.3 ± | 22.8 ± | 99.4 ± |
| 0.1% | before | | 0.8 | 0.2 | 3.3 | 0.1 |
| PS80 | nebulization | | | | | |
| | Flagellin after | Multimodal | 15.6 ± | 5.6 ± | 29.9 ± | 98.4 ± |
| | nebulization | | 4.2 | 0.4 | 7.6 | 1.4 |

The DLS analysis showed a low aggregation (submicronic particles) especially in acetate with PS80, for which the PDI was not multimodal and the percentages of monomer in intensity and mass were high. The results of histidine with PS80 before nebulization were not analyzable probably because of multiple submicronic particles in the sample. After nebulization of Flagellin in histidine, the percentages in mass and intensity of the monomer were the lowest.

The results showed that in all buffers w/o surfactant the aggregation of Flagellin after mesh-nebulization is high. The addition of PS80 reduces Flagellin aggregation in all buffers. The acetate and phosphate buffers appeared to be the most adapted to maintain the stability of Flagellin during mesh-nebulization. These buffers were selected to optimize further the formulation.

### Step 2: Determining the Optimal Surfactant and its Minimal Concentration to Stabilize Flagellin During Mesh-Nebulization

In this regard, nebulization stress was applied to Flagellin formulated in buffers with different types of surfactant and surfactant at different concentrations.

Flagellin was prepared at a concentration of 0.5 g/L. Acetate pH 5.5 and Phosphate pH 6.5 buffers were employed. First, the buffers were tested with Polysorbate 80 (PS80) at concentrations of 0.02%, 0.05%, 0.1%. For Phosphate buffer, lower concentrations of PS80 were also test including 0.01% and 0.05% with a new batch of Flagellin. For Acetate buffer, different surfactants were tested including Polysorbate 80 (PS80), Polysorbate 20 (PS20) and Poloxamer188

A nebulization stress was applied with a Solo (Aerogen) vibrating mesh nebulizer on 1 mL of Flagellin in the different formulations.

The degree of aggregation was measured using Flow Cell Microscopy (FCM) and Dynamic Light Scattering (DLS). The results are summarized in the Tables below

### Concentration of Surfactant

In acetate buffer, as analyzed by FCM, the concentration of 0.1% of PS80 was the more adapted to stabilize Flagellin as the concentration of total particle after nebulization was the smallest. However, the number of particles >2 μm was low for all formulation in presence of PS80. For Flagellin formulated with 0.1% of PS80, the PDI was low before nebulization and slightly increased after nebulization. For lower concentrations of PS80, the PDI were multimodal but the percentage in mass remained high and there were no major differences before and after nebulization.

| | | Particles | Particles >2 | Particles >10 | Particles >25 |
|---|---|---|---|---|---|
| | | total | μm/mL | μm/mL | μm/mL |
| Acetate | Flagellin before | 4023 ± | 715 ± | 22 ± | 5 ± |
| w/o PS80 | nebulization | 172 | 55 | 23 | 5 |
| | Flagellin after | 38430491 ± | 4150700 ± | 1628 ± | 0 ± |
| | nebulization | 14817201 | 3883187 | 3528 | 0 |
| Acetate | Flagellin before | 1643 ± | 99 ± | 5 ± | 3 ± |
| PS80 | nebulization | 217 | 33 | 5 | 5 |
| 0.02% | Flagellin after | 16804 ± | 440 ± | 38 ± | 5 ± |
| | nebulization | 11000 | 339 | 57 | 5 |
| Acetate | Flagellin before | 3436 ± | 348 ± | 13 ± | 0 ± |
| PS80 | nebulization | 72 | 137 | 12 | 0 |
| 0.05% | Flagellin after | 55649 ± | 465 ± | 54 ± | 0 ± |
| | nebulization | 62542 | 406 | 52 | 8 |
| Acetate | Flagellin before | 4195 ± | 976 ± | 36 ± | 0 ± |
| PS80 0.1% | nebulization | 794 | 379 | 63 | 0 |
| | Flagellin after | 3607 ± | 507 ± | 52 ± | 12 ± |
| | nebulization | 4849 | 701 | 61 | 14 |

| | | Global | | Monomer | | |
|---|---|---|---|---|---|---|
| | | | Z-average | Radius | | |
| | | PDI | (nm) | (nm) | % pd | % mass |
| Acetate | Flagellin | Multimodal | 5.6 ± | 10.6 ± | 97.4 ± | 99.9 ± |
| w/o PS80 | before | | 0.1 | 3.9 | 36.7 | 0.1 |
| | nebulization | | | | | |
| | Flagellin | 0.327 ± | 518.3 ± | | | |
| | after | 0.167 | 147 | | | |
| | nebulization | | | | | |
| Acetate | Flagellin | Multimodal | 34.8 ± | 5.3 ± | 33.9 ± | 96.6 ± |
| PS80 | before | | 7 | 1 | 13.1 | 1.6 |
| 0.02% | nebulization | | | | | |
| | Flagellin | Multimodal | 47.7 ± | 6.8 ± | 48.1 ± | 96.8 ± |
| | after | | 6.7 | 1.9 | 18.5 | 1.9 |
| | nebulization | | | | | |
| Acetate | Flagellin | Multimodal | 25.2 ± | 6.2 ± | 40.4 ± | 98.4 ± |
| PS80 | before | | 1 | 1.4 | 17.7 | 0.6 |
| 0.05% | nebulization | | | | | |
| | Flagellin | Multimodal | 22 ± | 6.1 ± | 37.7 ± | 98.6 ± |
| | after | | 5.2 | 0.8 | 8.8 | 0.6 |
| | nebulization | | | | | |
| Acetate | Flagellin | 0.119 ± | 5.1 ± | 5.3 ± | 23.7 ± | 99.9 ± |
| PS80 | before | 0.022 | 0.1 | 0.1 | 1.1 | 0.1 |
| 0.1% | nebulization | | | | | |
| | Flagellin | 0.314 ± | 5.6 ± | 5.3 ± | 23.9 ± | 100 ± |
| | after | 0.084 | 0.3 | 0.2 | 2.6 | 0.1 |
| | nebulization | | | | | |

In phosphate buffer, as observed by FCM, the concentration of 0.1% of PS8 was the more adapted to stabilize Flagellin. For lower concentrations of PS80, it is noticeable that the number of total particles before nebulization was greater than 15 000. But, there was no significant increase of total particles with PS8 at 0.02% before and after nebulization. The number of particles >2 μm was similar before and after nebulization for all PS8 concentrations. For all PS80 concentrations, Flagellin samples were multimodal (DLS). The percentage in mass of the monomer was higher for PS80 at 0.1%. For all concentrations, we observed a slight decrease of the percentage of the monomer in mass after nebulization as well as an increase of the Z-average except for the formulation with 0.1% of PS80 Hence, a moderate aggregation was observed when the concentration of PS80 was reduce under 0.1%.

| | | Particles | Particles >2 | Particles >10 | Particles >25 |
|---|---|---|---|---|---|
| | | total | μm/mL | μm/mL | μm/mL |
| Phosphate | Flagellin before | 3544 ± | 1361 ± | 40 ± | 5 ± |
| w/o PS80 | nebulization | 2353 | 1074 | 28 | 5 |
| | Flagellin after | 8297880 ± | 492779 ± | 425 ± | 19 ± |
| | nebulization | 4067956 | 776329 | 634 | 25 |
| Phosphate | Flagellin before | 17435 ± | 1691 ± | 0 ± | 11 ± |
| PS80 | nebulization | 792 | 215 | 12 | 9 |
| 0.02% | Flagellin after | 20531 ± | 1634 ± | 132 ± | 11 ± |
| | nebulization | 4710 | 1023 | 138 | 19 |
| Phosphate | Flagellin before | 18258 ± | 1775 ± | 102 ± | 8 ± |
| PS80 | nebulization | 1208 | 171 | 37 | 8 |
| 0.05% | Flagellin after | 46667 ± | 2478 ± | 188 ± | 11 ± |
| | nebulization | 38047 | 1424 | 191 | 25 |
| Phosphate | Flagellin before | 2630 ± | 729 ± | 96 ± | 27 ± |
| PS80 0.1% | nebulization | 2393 | 851 | 125 | 46 |
| | Flagellin after | 1156 ± | 858 ± | 188 ± | 26 ± |
| | nebulization | 1595 | 1192 | 260 | 37 |

| | | Global | | Monomer | | |
|---|---|---|---|---|---|---|
| | | | Z-average | Radius | | |
| | | PDI | (nm) | (nm) | % pd | % mass |
| Phosphate | Flagellin | Multimodal | 29.5 ± | 5.1 ± | 39.5 ± | 98.6 ± |
| w/o PS80 | before | | 0.9 | 0.5 | 8.3 | 0.4 |
| | nebulization | | | | | |
| | Flagellin | 0.208 ± | 324.2 ± | | | |
| | after | 0.07 | 19.3 | | | |
| | nebulization | | | | | |
| Phosphate | Flagellin | Multimodal | 41.4 ± | 7.0 ± | 39.4 ± | 94.5 ± |
| PS80 | before | | 1.7 | 3.9 | 17.9 | 3.5 |
| 0.02% | nebulization | | | | | |
| | Flagellin | Multimodal | 59.2 ± | 4.3 ± | 19.3 ± | 91.6 ± |
| | after | | 20.1 | 0.5 | 8.7 | 1.3 |
| | nebulization | | | | | |
| Phosphate | Flagellin | Multimodal | 24.2 ± | 5.8 ± | 37.4 ± | 97.4 ± |
| PS80 | before | | 1.6 | 0.5 | 5.8 | 1.5 |
| 0.05% | nebulization | | | | | |
| | Flagellin | Multimodal | 57.9 ± | 6.5 ± | 40.1 ± | 95.9 ± |
| | after | | 12.4 | 1.5 | 21.6 | 1.2 |
| | nebulization | | | | | |
| Phosphate | Flagellin | Multimodal | 11.6 ± | 5.3 ± | 22.8 ± | 99.4 ± |
| PS80 | before | | 0.8 | 0.2 | 3.3 | 0.1 |
| 0.1% | nebulization | | | | | |
| | Flagellin | Multimodal | 15.6 ± | 5.6 ± | 29.9 ± | 98.4 ± |
| | after | | 4.2 | 0.4 | 7.6 | 1.4 |
| | nebulization | | | | | |

Reduction of PS80 concentration in phosphate formulation

With this new batch, the high aggregation of Flagellin in phosphate without PS80 after nebulization was confirmed by FCM and DLS analysis. Indeed, millions of subvisibles particles were found by FCM and there were no more Flagellin monomers detectable by DLS. It also confirmed that addition of PS80 at 0.02% allowed to substantially reduce the aggregation as there was neither an increase of particles after nebulization nor a decrease of the percentage of monomer after nebulization. Interestingly, with this new batch Flagellin appeared to be less aggregated in phosphate with 0.02% PS80 than in the previous experiment in this formulation.

By reducing the PS80 concentration, we observed that, event at a very low concentration of PS80 (0.01 or 0.005%), the particles analyzed by FCM remained constant after nebulization and in a similar concentration than in formulation with 0.02% of PS8. The DLS results also indicated a low aggregation, as there were no monomer loss and the Z-average after nebulization remained closed to Z-average before nebulization. All PDI were multimodal or high.

| | | Particles | Particles >2 | Particles >10 | Particles >25 |
|---|---|---|---|---|---|
| | | total | μm/mL | μm/mL | μm/mL |
| Phosphate | Flagellin before | 4426 ± | 688 ± | 30 ± | 13 ± |
| w/o PS80 | nebulization | 1578 | 522 | 25 | 12 |
| | Flagellin after | 65173705 | 2784824 | 54 | 0 |
| | nebulization | | | | |
| | (n = 1) | | | | |
| Phosphate | Flagellin before | 7132 ± | 550 ± | 3 ± | 0 ± |
| PS80 | nebulization | 7788 | 620 | 5 | 0 |
| 0.005% | Flagellin after | 4174 ± | 430 ± | 27 ± | 0 ± |
| | nebulization | 1437 | 75 | 17 | 0 |
| Phosphate | Flagellin before | 5325 ± | 588 ± | 13 ± | 3 ± |
| PS80 | nebulization | 1937 | 58 | 9 | 5 |
| 0.01% | Flagellin after | 4111 ± | 583 ± | 30 ± | 0 ± |
| | nebulization | 2312 | 324 | 25 | 0 |
| Phosphate | Flagellin before | 5039 ± | 703 ± | 22 ± | 3 ± |
| PS80 | nebulization | 2359 | 430 | 17 | 5 |
| 0.02% | Flagellin after | 4083 ± | 430 ± | 32 ± | 11 ± |
| | nebulization | 2307 | 202 | 21 | 12 |

| | | Global | | Monomer | | |
|---|---|---|---|---|---|---|
| | | | Z-average | Radius | | |
| | | PDI | (nm) | (nm) | % pd | % mass |
| Phosphate | Flagellin | Multimodal | 7.8 ± | 4.5 ± | 36.4 ± | 99.9 ± |
| w/o PS80 | before | | 0.1 | 0.0 | 7.6 | 0.1 |
| | nebulization | | | | | |
| | Flagellin | 0.219 | 340.9 | | | |
| | after | | | | | |
| | nebulization | | | | | |
| Phosphate | Flagellin | Multimodal | 5.9 ± | 5.2 ± | 44.2 ± | 100 ± |
| PS80 | before | | 0.3 | 0.1 | 1.8 | 0.0 |
| 0.005% | nebulization | | | | | |
| | Flagellin | 0.483 ± | 4.9 ± | 5.6 ± | 51.4 ± | 99.8 ± |
| | after | 0.088 | 0.4 | 0.3 | 3.6 | 0.3 |
| | nebulization | | | | | |
| Phosphate | Flagellin | Multimodal | 5.5 ± | 5.0 ± | 33.9 ± | 99.9 ± |
| PS80 | before | | 0.4 | 0.3 | 12.8 | 0.1 |
| 0.01% | nebulization | | | | | |
| | Flagellin | 0.409 ± | 5.1 ± | 5.3 ± | 38.4 ± | 99.9 ± |
| | after | 0.123 | 0.2 | 0.6 | 10.9 | 0.1 |
| | nebulization | | | | | |
| Phosphate | Flagellin | 0.474 ± | 5.4 ± | 5.1 ± | 29.7 ± | 99.9 ± |
| PS80 | before | 0.1 | 0.1 | 0.2 | 4.1 | 0.1 |
| 0.02% | nebulization | | | | | |
| | Flagellin | Multimodal | 5.5 ± | 5.1 ± | 31.6 ± | 100 ± |
| | after | | 0.1 | 0.2 | 4.2 | 0.0 |
| | nebulization | | | | | |

### Type of Surfactants

Regarding FCM analysis, the number of total particles (subvisible) after nebulization was higher with PS20 and Poloxamer than PS80. For particles >2 μm, a high aggregation was also observed with Poloxamer.

The DLS analysis of Flagellin after nebulization was not usable with Poloxamer and limited with PS20 (n=1/3), probably because of the presence of too many submicronic particles.

| | | Particles | Particles >2 | Particles >10 | Particles >25 |
|---|---|---|---|---|---|
| | | total | μm/mL | μm/mL | μm/mL |
| Acetate | Flagellin before | 1643 ± | 99 ± | 5 ± | 3 ± |
| PS80 | nebulization | 217 | 33 | 5 | 5 |
| 0.02% | Flagellin after | 16804 ± | 440 ± | 38 ± | 5 ± |
| | nebulization - | 11000 | 339 | 57 | 5 |
| | buffer | | | | |
| Acetate | Flagellin before | 14748 ± | 752 ± | 51 ± | 17 ± |
| PS20 | nebulization | 1022 | 66 | 12 | 19 |
| 0.02% | Flagellin after | 42933 ± | 194 ± | 5 ± | 3 ± |
| | nebulization | 52660 | 171 | 33 | 16 |
| Acetate | Flagellin before | 3938 ± | 246 ± | 0 ± | 0 ± |
| Poloxamer | nebulization | 214 | 28 | 0 | 0 |
| 0.02% | Flagellin after | 1448465 ± | 11913 ± | 94 ± | 3 ± |
| | nebulization | 2364676 | 18815 | 76 | 7 |

| | | Global | | Monomer | | |
|---|---|---|---|---|---|---|
| | | | Z-average | Radius | | |
| | | PDI | (nm) | (nm) | % pd | % mass |
| Acetate | Flagellin | Multimodal | 34.8 ± | 5.3 ± | 33.9 ± | 96.6 ± |
| PS80 | before | | 7 | 1 | 13.1 | 1.6 |
| 0.02% | nebulization | | | | | |
| | Flagellin | Multimodal | 47.7 ± | 6.8 ± | 48.1 ± | 96.8 ± |
| | after | | 6.7 | 1.9 | 18.5 | 1.9 |
| | nebulization | | | | | |
| Acetate | Flagellin | Multimodal | 18.9 ± | 5.6 ± | 41.1 ± | 99.4 ± |
| PS20 | before | | 1.5 | 0.9 | 11.4 | 0.1 |
| 0.02% | nebulization | | | | | |
| | Flagellin | Multimodal | 51.3 | 4.7 | 24.6 | 98.3 |
| | after | | | | | |
| | nebulization | | | | | |
| | (n = ⅓) | | | | | |
| Acetate | Flagellin | Multimodal | 23.3 ± | 4.7 ± | 35.4 ± | 98.8 ± |
| Poloxamer | before | | 3.6 | 0.2 | 6.1 | 0.2 |
| 0.02% | nebulization | | | | | |
| | Flagellin | NA | | | | |
| | after | | | | | |
| | nebulization | | | | | |

The results showed that a concentration of 0.1% of PS80 may be more adapted to limit the aggregation of Flagellin. However, for both phosphate and acetate, 0.02% of PS80 reduced significantly the number of subvisible and submicronic particles as compared to the buffer w/o surfactant. It appeared that very low concentration of PS80 (0.05%) was enough to stabilize the Flagellin in phosphate during mesh-nebulization. The comparison of surfactant showed that PS80 was better than PS20 and Poloxamer to stabilize Flagellin during mesh-nebulization.

### Step 3: Impact of Flagellin Concentration on the Stability of Flagellin During Mesh-Nebulization

In this regard, the nebulization stress was applied to flagellin formulated at 4 different concentrations.

The Flagellin was prepared in phosphate pH 6.5 buffer with 0.02% of PS80 at concentrations of 0.1 g/L, 0.5 g/L, 1 g/L and 3 g/L

A nebulization stress was applied with a Solo (Aerogen) vibrating mesh nebulizer on 1 mL of Flagellin in the different formulations.

The degree of aggregation was measured using Flow Cell Microscopy (FCM) and Dynamic Light Scattering (DLS). The results are summarized in the Tables below

| | | Particles | Particles >2 | Particles >10 | Particles >25 |
|---|---|---|---|---|---|
| | | total | μm/mL | μm/mL | μm/mL |
| Flagellin | Flagellin before | 1966 ± | 121 ± | 3 ± | 0 ± |
| 0.1 g/L | nebulization | 149 | 28 | 5 | 0 |
| Phosphate | Flagellin after | 437 ± | 8 ± | 3 ± | 3 ± |
| PS80 | nebulization | 606 | 18 | 4 | 4 |
| 0.02% | | | | | |
| Flagellin | Flagellin before | 3070 ± | 389 ± | 19 ± | 3 ± |
| 0.5 g/L | nebulization | 176 | 94 | 12 | 5 |
| Phosphate | Flagellin after | 10292 ± | 333 ± | 19 ± | 8 ± |
| PS80 | nebulization | 19967 | 558 | 52 | 18 |
| 0.02% | | | | | |
| Flagellin | Flagellin before | 1649 ± | 261 ± | 19 ± | 0 ± |
| 1 g/L | nebulization | 860 | 184 | 17 | 0 |
| Phosphate | Flagellin after | 5188 ± | 1097 ± | 68 ± | 0 ± |
| PS80 | nebulization | 8056 | 2077 | 91 | 0 |
| 0.02% | | | | | |
| Flagellin | Flagellin before | 1487 ± | 202 ± | 5 ± | 3 ± |
| 3 g/L | nebulization | 101 | 69 | 5 | 5 |
| Phosphate | Flagellin after | 2738 ± | 297 ± | 0 ± | 0 ± |
| PS80 | nebulization | 4046 | 501 | 0 | 0 |
| 0.02% | | | | | |

Overall, the FCM analysis showed a low aggregation (subvisible particles) degree of Flagellin when it was formulated at different concentrations. The number of total particles per mL was slightly higher at 0.5 g/L and 1 g/L as compared to 0.1 g/L and 3 g/L. Regarding particles >2 μm/mL, an increase after nebulization was observed only at a concentration of 1 g/L.

For all samples analyzed by DLS, the PDI were multimodal. There was no decrease of the percentage in mass of Flagellin monomer.

| | | Global | | Monomer | | |
|---|---|---|---|---|---|---|
| | | | Z-average | Radius | | |
| | | PDI | (nm) | (nm) | % pd | % mass |
| Flagellin | Flagellin | Multimodal | 6.8 ± | 6 ± | 38.3 ± | 99.9 ± |
| 0.1 g/L | before | | 0.1 | 0.4 | 4.9 | 0.1 |
| Phosphate | nebulization | | | | | |
| PS80 | Flagellin | Multimodal | 15.3 ± | 5.4 ± | 38.5 ± | 99.3 ± |
| 0.02% | after | | 8.2 | 0.3 | 5.5 | 0.5 |
| | nebulization | | | | | |
| Flagellin | Flagellin | Multimodal | 50.7 ± | 4.7 ± | 19.9 ± | 96.0 ± |
| 0.5 g/L | before | | 3.8 | 0.3 | 8.4 | 0.5 |
| Phosphate | nebulization | | | | | |
| PS80 | Flagellin | Multimodal | 17.3 ± | 5.8 ± | 39.3 ± | 98.9 ± |
| 0.02% | after | | 5.6 | 0.5 | 8 | 1.1 |
| | nebulization | | | | | |
| Flagellin | Flagellin | Multimodal | 25.3 ± | 5 ± | 26.3 ± | 98.0 ± |
| 1 g/L | before | | 0.9 | 0.6 | 13.3 | 0.5 |
| Phosphate | nebulization | | | | | |
| PS80 | Flagellin | Multimodal | 15.3 ± | 5.4 ± | 38.5 ± | 99.3 ± |
| 0.02% | after | | 16.1 | 1.1 | 18.9 | 1.7 |
| | nebulization | | | | | |
| Flagellin | Flagellin | Multimodal | 43.3 ± | 5.5 ± | 39 ± | 96.2 ± |
| 3 g/L | before | | 1.2 | 1.7 | 14.4 | 2.5 |
| Phosphate | nebulization | | | | | |
| PS80 | Flagellin | Multimodal | 15.3 ± | 5.4 ± | 38.5 ± | 99.3 ± |
| 0.02% | after | | 1.8 | 0.2 | 5 | 0.3 |
| | nebulization | | | | | |

The results showed that the concentration of Flagellin did not influence much the stability of Flagellin during mesh-nebulization.

### Complementary Analysis for Selected Formulations

Based on the formulation study, two formulations were selected for their capacity to maintain the stability of Flagellin during mesh-nebulization and their compatibility for the pulmonary environment: Acetate pH 5.5 with 0.02% of PS80 and Phosphate pH 6.5 with 0.02% of PS80.

For these formulations, additional assay to analyze the effect of the formulations on the aerosol properties and activity of Flagellin

The aerosol was characterized by laser diffraction to obtain the droplet size distribution of Flagellin nebulized in Acetate or Phosphate in comparison to NaCl 0.9% alone.

The activity of Flagellin was evaluated by calculating the EC₅₀ of Flagellin in the different formulations before and after nebulization

The results are summarized in the Tables below

### Aerosol Properties

| | VMD (μm) | <5 μm (%) | 0.5-3 μm (%) |
|---|---|---|---|
| NaCl | 4.0 | 60.0 | 33.0 |
| Flagellin 0.5 g/L Acetate | 4.2 | 61.4 | 31.3 |
| PS80 0.02% | | | |
| Flagellin 0.5 g/L Phosphate | 4.3 | 59.7 | 29.7 |
| PS80 0.02% | | | |

The formulations did not influence the aerosol properties that remained compatible with lung delivery. The fine particles fraction (particles <5 μm) is around 60% meaning that a high proportion of the aerosol reaches the lungs.

### Activity

| | | EC₅₀ (μg/mL) |
|---|---|---|
| Flagellin | Flagellin before | 0.00211 |
| 0.5 g/L | nebulization | |
| Acetate | Flagellin after | 0.00278 |
| PS80 0.02% | nebulization | |
| Flagellin | Flagellin before | 0.00534 |
| 0.5 g/L | nebulization | |
| Phosphate | after Flagellin | 0.00247 |
| PS80 0.02% | nebulization | |

There was no significant change of the EC₅₀ after nebulization of Flagellin and the EC₅₀ were comparable between acetate and phosphate buffers. Hence, the Flagellin potency on TLR5 activation was maintained after nebulization in both formulation.

### Example 2

In complement to Example 1, Step 3: Impact of flagellin concentration on the stability of flagellin during mesh-nebulization

Characterize Flagellin Biological Activity, after Mesh-Nebulization, at Different Concentrations in the Optimal Formulation:
To fit with human application, with the active ingredient comprised between 0.01 to 0.5 mg in the lungs.
Stability of FLAMOD during nebulization after dilution in the selected buffer Biological activity
Evaluation of Flagellin biological activity from 10 μg/mL to 500 μg/mL in Phosphate 10 mM pH6.5+NaCl 145 mM+PS80 0.02%
   (10 nebulizations per concentration, analyzed in triplicate)
   Using a cell reporter assay (HEK-Dual™ hTLR5)

### Results:

Evaluation of Flagellin biological activity from 10 μg/mL to 500 μg/mL in (10 nebulizations per concentration, analyzed in triplicate).

| | | No | 500 | 250 | 100 | 50 | 25 | 10 |
|---|---|---|---|---|---|---|---|---|
| | | Nebulization | μg/mL | μg/mL | μg/mL | μg/mL | μg/mL | μg/mL |
| EC50 | MEAN | 0.304 | 0.412 | 0.280 | 0.464 | 0.395 | 0.334 | 0.363 |
| (ng/mL) | SD | 0.293 | 0.363 | 0.245 | 0.186 | 0.121 | 0.111 | 0.131 |

The activity of Flagellin was not significantly modified after nebulization whatever the concentration of Flagellin in the optimal formulation.

1. An aerosol composition comprising droplets comprising a liquid formulation, wherein the liquid formulation comprises
(i) a flagellin polypeptide,
(ii) a buffering agent selected from the group consisting of acetate, phosphate and combinations thereof,
(iii) a surfactant consisting of polysorbate, and
(iv) an aqueous medium; and
wherein the liquid formulation has a pH which is equal to or lower than 8.

2. The aerosol composition of claim 1, wherein the surfactant is polysorbate 80.

3. The aerosol composition of claim 1, wherein the buffering agent is acetate, and the liquid formulation has a pH which is lower than 5.8 or lower than 5.5.

4. The aerosol composition of, wherein the buffering agent is phosphate, and the liquid formulation has a pH which is lower than 6.8 or lower than 6.5.

5. The aerosol composition of claim 1, wherein the concentration of the surfactant in the liquid formulation is equal to or lower than 0.1% (w/v) or equal to or lower than 0.02% (w/v) or equal to or lower than 0.005% (w/v).

6. The aerosol composition of claim 1, wherein the flagellin polypeptide comprises: a) a N-terminal peptide having at least 90% amino acid identity with the amino acid sequence starting from the amino acid residue located at position 1 of SEQ ID NO:3 and ending at an amino acid residue selected from the group consisting of any one of the amino acid residues located at positions 99 to 173 of SEQ ID NO:3; and b) a C-terminal peptide having at least 90% amino acid identity with the amino acid sequence starting at an amino acid residue selected from the group consisting of any one of the amino acid residues located at positions 401 to 406 of SEQ ID NO:3 and ending at the amino acid residue located at position 494 of SEQ ID NO:3, wherein: the N-terminal peptide is directly linked to the C-terminal peptide, or the N-terminal peptide and the C-terminal peptide are indirectly linked, one to the other, through a spacer chain.

7. The aerosol composition of claim 6, wherein N-terminal and C-terminal peptides consist of the amino acid sequences 1-173 and 401-494 of SEQ ID NO:3, respectively.

8. The aerosol composition of claim 6, wherein N-terminal peptide and the C-terminal peptide are indirectly linked, one to the other, through an intermediate spacer chain consisting of a NH₂-Gy-Aa-Aa-GIy-COOH (SEQ ID NO:4) peptide sequence.

9. The aerosol composition of claim 8, wherein the flagellin polypeptide is the polypeptide having the amino acid sequence of SEQ ID No 5.

10. A method of preparing an aerosol composition comprising droplets comprising a liquid formulation, said method comprising the steps:
(i) providing a liquid formulation as defined in claim 1, and
(ii) nebulizing the liquid formulation provided in step (i) by means of a nebulizer, thereby preparing the aerosol.

11. The method of claim 10, wherein, optionally, said method further comprises, between steps (i) and (ii), the steps of:
(ia) lyophilizing the liquid formulation provided in step (i), thereby providing a lyophilized powder, and
(ib) reconstituting the liquid formulation provided in step (i) by adding an appropriate amount of an aqueous medium to the lyophilized powder provided in step (ia).

12. or the A method of delivering a flagellin polypeptide to the lungs of a subject, comprising administering the aerosol composition of claim 1 by inhalation or the liquid formulation of claim 1 by inhalation via a nebulizer.

13. A method of treating or preventing an infectious disease in the lung of a subject, comprising administering to the subject the aerosol composition of claim 1 by inhalation or the liquid formulation of claim 1 by inhalation via a nebulizer, wherein, optionally, at least one antibiotic is administered with the liquid formulation or the aerosol composition.

14. A kit comprising
(i) a container comprising the liquid formulation as defined in claim 1 or a powder obtainable by lyophilization of the liquid formulation, and
(ii) a nebulizer.

15. A method of preparing an aerosol composition comprising
(i) providing the liquid formulation of claim 1, and
(ii) nebulizing the liquid formulation provided in step (i) by means of a nebulizer, thereby preparing the aerosol.

16. A method of increasing the stability of a flagellin polypeptide upon nebulization of a liquid formulation comprising including a buffering agent selected from the group consisting of acetate, phosphate and combinations thereof and a surfactant in the liquid formulation prior to nebulization.


SEQUENCE LISTING (5 sequences)

SEQ ID NO: 1 - protein, 585 aa, Escherichia coli
MAQVINTNSLSLITQNNINKNQSALSSSIERLSSGLRINSAKDDAAGQAIANRFTSNIKGLTQAARNANDGISVAQTTEGALSEINNNLQRIRELTVQATTGTNSDSDLDSIQDEIKSRLDEIDRVSGQTQFNGVNVLAKDGSMKIQVGANDGETITIDLKKIDSDTLGLNGFNVNGKGTITNKAATVSDLTSAGAKLNTTTGLYDLKTENTLLTTDAAFDKLGNGDKVTVGGVDYTYNAKSGDFTTTKSTAGTGVDAAAQAADSASKRDALAATLHADVGKSVNGSYTTKDGTVSFETDSAGNITIGGSQAYVDDAGNLTTNNAGSAAKADMKALLKAASEGSDGASLTFNGTEYTIAKATPATTTPVAPLIPGGITYQATVSKDVVLSETKAAAATSSITFNSGVLSKTIGFTAGESSDAAKSYVDDKGGITNVADYTVSYSVNKDNGSVTVAGYASATDTNKDYAPAIGTAVNVNSAGKITTETTSAGSATTNPLAALDDAISSIDKFRSSLGAIQNRLDSAVTNLNNTTTNLSEAQSRIQDADYATEVSNMSKAQIIQQAGNSVLAKANQVPQQVLSLLQG

SEQ ID NO: 2 - protein, 495 aa, Salmonella typhimurium [sequence omitted]

SEQ ID NO: 3 - protein, 494 aa, Salmonella typhimurium
AQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGEVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 4 - protein, 4 aa, Artificial
  Synthetic linker
GAAG

SEQ ID NO: 5 - protein, 273 aa, Artificial
  Synthetic FLAMOD
MKAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNGAAGATTTENPLQKIDAALAQVDTLRSDLGAVQNRFNSAITNLGNTVNNLTSARSRIEDSDYATEVSNMSRAQILQQAGTSVLAQANQVPQSVLSLLR